(19) United States
(12) Patent Application Publication
(10) Pub. No.: US 2014/0049770 A1
(43) Pub. Date: Feb. 20, 2014

(54) DETERMINING ABSORPTION COEFFICIENTS IN A PHOTOACOUSTIC SYSTEM

(71) Applicants: Youzhi Li, Longmont, CO (US); Bo Chen, Louisville, CO (US)

(72) Inventors: Youzhi Li, Longmont, CO (US); Bo Chen, Louisville, CO (US)

(73) Assignee: Nellcor Puritan Bennett LLC, Boulder, CO (US)

(21) Appl. No.: 13/585,960

(22) Filed: Aug. 15, 2012

(51) Int. Cl. G01N 33/49; G01N 21/00

(52) U.S. Cl. 356/40; 356/72

(57) ABSTRACT

A physiological monitoring system may use photoacoustic sensing to determine physiological information of a subject. The photoacoustic monitoring system may use a light source, such as a modulated continuous wave laser diode, to provide a frequency modulated photonic signal (e.g., a chirp signal) to the subject. An acoustic detector may be used to detect an acoustic pressure signal from the subject. The acoustic pressure signal may include two components corresponding to two wavelengths of light in the photonic signal. A signal ratio may be calculated based on the two components. The photoacoustic monitoring system may use the signal ratio to calculate one or more absorption coefficients. The photoacoustic monitoring system may use the one or more absorption coefficients to determine additional physiological information such as hemoglobin concentration, blood oxygen saturation, and temperature.

The present disclosure relates to determining absorption coefficients, and more particularly relates to determining absorption coefficients used in part to calculate physiological parameters in a photoacoustic system.

### SUMMARY

Systems and methods are provided for determining absorption coefficients and physiological parameter of a subject using a photoacoustic system. The system may determine optical absorption coefficients of a subject by comparing elements of photoacoustic signals generated at multiple wavelengths by the subject in response to a light signal. The system may include a light source configured to provide a frequency modulated light signal at a first wavelength and a second wavelength. The system may modulate the light signal with, for example, a linear chirp. The system may include at least one acoustic detector, such as a piezoelectric ultrasound detector, configured to detect an acoustic pressure signal from the subject. The acoustic pressure signal may be caused by absorption of at least some of the photonic signal by the subject. The acoustic pressure signal may include a first component corresponding to the first wavelength and a second component corresponding to the second wavelength.

The system may calculate at least one photoacoustic signal ratio based on the first component and the second component. For example, the system may determine a normalized spectral ratio of two photoacoustic signals. In some embodiments, the system may perform processing in the frequency domain and the time domain. The system may modify signals to isolate structural elements in the subject. For example, the system may isolate a portion of a time-domain signal related to a blood vessel, perform a transform of the isolated signal into the frequency domain, and calculate a normalized spectral ratio in the frequency domain. The system may determine at least one optical absorption coefficient based on the photoacoustic signal ratio. For example, the system may fit one or more equations to the spectral ratio and extract parameters that may be related to physiological parameters. For example, the system may determine an optical absorption coefficient and/or a fluence ratio of the two signals. The system may determine one or more physiological parameters based on the optical absorption coefficient. For example, the system may determine a blood oxygen saturation parameter, temperature parameter, blood pressure parameter, cardiac output parameter, or a combination thereof.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative physiological monitoring system in accordance with some embodiments of the present disclosure;

FIG. 2 is a block diagram of an illustrative sensor unit of the physiological monitoring system of FIG. 1, which may be coupled to a subject, in accordance with some embodiments of the present disclosure;

FIG. 3 is a block diagram of an illustrative signal processing system in accordance with some embodiments of the present disclosure;

FIG. 4 is an illustrative photoacoustic arrangement in accordance with some embodiments of the present disclosure;

FIG. 5 is an illustrative plot of frequency-domain photoacoustic signals in accordance with some embodiments of the present disclosure;

FIG. 6 is an illustrative plot of the ratio of two frequency-domain photoacoustic signals in accordance with some embodiments of the present disclosure;

FIG. 7 is a flow diagram showing illustrative steps for determining a physiological parameter in accordance with some embodiments of the present disclosure;

FIG. 8 shows an illustrative indicator dilution photoacoustic arrangement in accordance with some embodiments of the present disclosure;

FIG. 9 shows an illustrative indicator arrangement 800 in accordance with some embodiments of the present disclosure;

FIG. 10 shows a plot of an illustrative photoacoustic signal, including a response to an isotonic indicator, in accordance with some embodiments of the present disclosure; and

FIG. 11 shows an illustrative plot of total hemoglobin concentration as isotonic and hypertonic indicators pass a photoacoustic detection site in accordance with some embodiments of the present disclosure.

### DETAILED DESCRIPTION OF THE FIGURES

Photoacoustics (or “optoacoustics”) or “the photoacoustic effect” (or “optoacoustic effect”) refers to the phenomenon in which one or more wavelengths of light are presented to and absorbed by one or more constituents of an object, thereby causing an increase in kinetic energy of the one or more constituents, which causes an associated pressure response within the object. Particular modulations or pulsing of the incident light, along with measurements of the corresponding pressure response in, for example, tissue of the subject, may be used for physiological parameter determination, medical imaging, or both. For example, the concentration of a constituent, such as hemoglobin (e.g., oxygenated, deoxygenated and/or total hemoglobin) may be determined using photoacoustic analysis. In a further example, one or more hemodynamic parameters such as cardiac output (CO), intrathoracic blood volume (ITBV), intrathoracic circulatory volume (ITCV), global end-diastolic volume (GEDV), pulmonary circulatory volume (PCV), extravascular lung water (EVLW), and/or any other suitable hemodynamic parameters may be determined using photoacoustic analysis and indicator dilution techniques. In a further example, physiological parameters such as temperature may be determined using photoacoustic analysis. In some embodiments, the system may use multiple wavelengths of light from multiple light sources to determine physiological parameters, for example, blood oxygen concentration. In some embodiments, the system may use a single light source to determine physiological parameters, for example, temperature or cardiac output.

Hemoglobin is understood herein to be a complex protein carried in the bloodstream of a subject that is typically involved in transporting oxygen. Hemoglobin can carry oxygen by varying the oxidation state of an iron atom within the hemoglobin protein. Hemoglobin can be found in at least two states including oxyhemoglobin and deoxyhemoglobin. Oxyhemoglobin is understood to represent the oxygenated state of hemoglobin. Oxyhemoglobin is involved in the process of transporting oxygen molecules from, for example, the lungs to various muscles, organs and other tissues of the subject. Deoxyhemoglobin is understood to be the deoxygenated state of hemoglobin, which occurs, for example, after a molecule of oxyhemoglobin releases oxygen for delivery to a muscle, organ, or other tissue of the subject.

Cardiac output, as used herein, is understood to include the amount of blood pumped by the heart over a particular time interval. One clinical method of determining cardiac output includes rapidly injecting an amount of diluent (e.g., saline) into a blood vessel and subsequently measuring the time-dependent change in blood concentration (i.e., indicator dilution curve) downstream from the diluent injection site. A relatively fast return from the diluted state to a baseline blood concentration may indicate a high cardiac output, while a slow return may indicate low cardiac output. A photoacoustic system may be used to monitor blood solute concentration and determine cardiac output.

Temperature may be a desired physiological parameter. As used herein, temperature is understood to be the internal or external temperature of a body structure. For example, in a clinical setting, the internal temperature of a patient recovering from hypothermia or undergoing cryotherapy may be desired to adjust treatment parameters. For many tissues, the Grüneisen parameter may be highly sensitive to changes in temperature, while optical absorption parameters may be relatively insensitive to temperature changes. The use of photoacoustic temperature measurements may allow for accurate, non-invasive internal temperature determination.

A photoacoustic system may include a photoacoustic sensor that is placed at a site on a subject, typically a cheek, sublingual area, temple, neck, wrist, palm, fingertip, toe, forehead or earlobe, or in the case of a neonate, across a foot. In some embodiments, the photoacoustic sensor can be placed anywhere where an artery or vessel is accessible noninvasively. The photoacoustic system may use a light source, and any suitable light guides (e.g., fiber optics), to provide light to the subject's tissue, or a combination of tissue thereof (e.g., organs) and an acoustic detector to sense the pressure response of the tissue induced by light absorption. Tissue may include muscle, fat, blood, blood vessels, and/or any other suitable tissue types. In some embodiments, the light source may be a laser or laser diode, operated in pulsed or continuous wave (CW) mode. In some embodiments, the acoustic detector may be an ultrasound detector, which may be suitable to detect pressure fluctuations arising from the constituent's absorption of the incident light of the light source.

In some embodiments, the light from the light source may be focused, shaped, or otherwise spatially modulated to illuminate a particular region of interest. In some arrangements, photoacoustic monitoring may allow relatively higher spatial resolution than line of sight optical techniques (e.g., path integrated absorption measurements). The enhanced spatial resolution of the photoacoustic technique may allow for imaging, scalar field mapping, and other spatially resolved results, in 1, 2, or 3 spatial dimensions. The acoustic response to the photonic excitation may radiate from the illuminated region of interest, and accordingly may be detected at multiple positions.

The photoacoustic system may measure the pressure response that is received at the acoustic sensor as a function of time. The photoacoustic system may also include sensors at multiple locations. A signal representing pressure versus time or a mathematical manipulation of this signal (e.g., a scaled version thereof, etc.) may be referred to as the photoacoustic signal. The photoacoustic signal may be derived from a detected acoustic pressure signal by selecting a suitable subset of points of an acoustic pressure signal. The photoacoustic signal may also be derived using an envelope technique on the absolute values of the acoustic pressure signal. The photoacoustic signal may be used to calculate any of a number of physiological parameters, including oxygen saturation and a concentration of a blood constituent (e.g., oxyhemoglobin), at a particular spatial location. In some embodiments, photoacoustic signals from multiple spatial locations may be used to construct an image (e.g., imaging blood vessels) or a scalar field (e.g., a hemoglobin concentration field). As used herein, blood vessels are understood to include the veins, arteries, and capillaries of a subject.

In some applications, the light passed through the tissue is selected to be of one or more wavelengths that are absorbed by the constituent in an amount representative of the amount of the constituent present in the tissue. The absorption of light passed through the tissue varies in accordance with the amount of the constituent in the tissue. For example, Red and/or infrared (IR) wavelengths may be used because highly oxygenated blood will absorb relatively less Red light and more IR light than blood with a lower oxygen saturation.

Any suitable light source may be used, and characteristics of the light provided by the light source may be controlled in any suitable manner. In some embodiments, a pulsed light source may be used to provide relatively short-duration pulses (e.g., nano-second pulses) of light to the region of interest. Accordingly, the use of a pulse light source may result in a relatively broadband acoustic response (e.g., depending on the pulse duration). The use of a pulsed light source will be referred to herein as the “Time Domain Photoacoustic” (TD-PA) technique. A convenient starting point for analyzing a TD-PA signal is given by Eq. 1:

p(z)=Γμₐφ(z) (1)

under conditions where the irradiation time is small compared to the characteristic thermal diffusion time determined by the properties of the specific tissue type. Referring to Eq. 1, p(z) is the photoacoustic signal (indicative of the maximum induced pressure rise, derived from an acoustic signal) at spatial location z indicative of acoustic pressure, Γ is the dimensionless Grüneisen parameter of the tissue, μₐ is the absorption coefficient of the tissue at location z (or constituent thereof) to the incident light, and 4(z) is the optical fluence at spatial location z. The Grüneisen parameter is a dimensionless description of thermoelastic effects, and may be illustratively formulated by Eq. 2:

$$Γ = \frac{β {c}_{a}^{2}}{{C}_{P}} \qquad ( 2 )$$

where cₐ is the speed of sound in the tissue, β is the isobaric volume thermal expansion coefficient, and C_{P} is the specific heat at constant pressure. In some circumstances, the optical fluence, at spatial location z (within the subject's tissue) of interest may be dependent upon the light source, the location itself (e.g., the depth), and optical properties (e.g., scattering coefficient, absorption coefficient, or other properties) along the optical path. For example, Eq. 3 provides an illustrative expression for the attenuated optical fluence at a depth z:

φ(z)=φ₀e^{-μ}_{^{eff}}^{x} (3)

where φ₀ is the optical fluence from the light source incident at the tissue surface, z is the path length (i.e., the depth into the tissue in this example), and μ_{eff} is an effective attenuation coefficient of the tissue along the path length in the tissue in this example.

In some embodiments, a more detailed expression or model may be used rather than the illustrative expression of Eq. 3. In some embodiments, the actual pressure encountered by an acoustic detector may be proportional to Eq. 1, as the focal distance and solid angle (e.g., face area) of the detector may affect the actual measured photoacoustic signal. In some embodiments, an ultrasound detector positioned relatively farther away from the region of interest will encounter a relatively smaller acoustic pressure. For example, the peak acoustic pressure signal p_{d} received at a circular area A_{d} positioned at a distance R from the illuminated region of interest may be given by Eq. 4:

p_{d}=p(z)f(rₛ,R,A_{d},a) (4)

where rₛ is the radius of the illuminated region of interest (and typically rₛ<R), p(z) is given by Eq. 1, and a is related to the acoustic attenuation. In some embodiments, the detected acoustic pressure amplitude may decrease as the distance R increases (e.g., for a spherical acoustic wave).

In some embodiments, a modulated CW light source may be used to provide a photonic excitation of a tissue constituent to cause a photoacoustic response in the tissue. The CW light source may be intensity modulated at one or more characteristic frequencies. The use of a CW light source, intensity modulated at one or more frequencies, will be referred to herein as the “Frequency Domain Photoacoustic” (FD-PA) technique. Although the FD-PA technique may include using frequency domain analysis, the technique may use time domain analysis, wavelet domain analysis, or any other suitable analysis, or any combination thereof. Accordingly, the term “frequency domain” as used in “FD-PA” refers to the frequency modulation of the photonic signal, and not to the type of analysis used to process the photoacoustic response.

Under some conditions, the acoustic pressure p(R,t) at detector position R at time t, may be shown illustratively by Eq. 5:

$$p ( R , t ) ∼ \frac{{p}_{0} ( {r}_{0} , ω )}{R} {e}^{- iω ( t - τ )} \qquad ( 5 )$$

where r₀ is the position of the illuminated region of interest, ω is the angular frequency of the acoustic wave (caused by modulation of the photonic signal at frequency ω), R is the distance between the illuminated region of interest and the detector, and τ is the travel time delay of the wave equal to R/cₐ, where cₐ is the speed of sound in the tissue. The FD-PA spectrum p₀(r₀,ω) of acoustic waves is shown illustratively by Eq. 6:

$${p}_{0} ( {r}_{0} , ω ) = \frac{{Γμ}_{a} φ ( {r}_{0} )}{2 ( {μ}_{a} {c}_{a} - iω )} \qquad ( 6 )$$

where μₐcₐ represents a characteristic frequency (and corresponding time scale) of the tissue.

In some embodiments, a FD-PA system may temporally vary the characteristic modulation frequency of the CW light source, and accordingly the characteristic frequency of the associated acoustic response. For example, the FD-PA system may use linear frequency modulation (LFM), either increasing or decreasing with time, which is sometimes referred to as “chirp” signal modulation. Shown in Eq. 7 is an illustrative expression for a sinusoidal chirp signal r(t)):

$$r ( t ) = cos ( t ( {ω}_{0} + \frac{b}{2} t ) ) \qquad ( 7 )$$

where ω₀ is a starting angular frequency, and b is the angular frequency scan rate. Any suitable range of frequencies (and corresponding angular frequencies) may be used for modulation such as, for example, 1-5 MHz, 200-800 kHz, or other suitable range, in accordance with the present disclosure. In some embodiments, signals having a characteristic frequency that changes as a nonlinear function of time may be used. Any suitable technique, or combination of techniques thereof, may be used to analyze a FD acoustic pressure signal. Two such exemplary techniques, a correlation technique and a heterodyne mixing technique, will be discussed below as illustrative examples.

In some embodiments, the correlation technique may be used to determine the travel time delay of the FD-PA signal. In some embodiments, a matched filtering technique may be used to process a photoacoustic signal. As shown in Eq. 8:

$${B}_{s} ( t - τ ) = \frac{1}{2 π} {\int }_{- ∞}^{∞} H ( ω ) S ( ω ) {e}^{i ω t} d ω \qquad ( 8 )$$

Fourier transforms (and inverse transforms) are used to calculate the filter output Bₛ(t−τ), in which H(ω) is the filter frequency response, S(ω) is the Fourier transform of the photoacoustic signal s(t), and τ is the phase difference between the filter and signal. In some circumstances, the filter output of expression of Eq. 8 may be equivalent to an autocorrelation function. Shown in Eq. 9:

$$S ( ω ) = \frac{1}{2 π} {\int }_{- ∞}^{∞} s ( t ) {e}^{- i ω t} d t \qquad ( 9 )$$

is an expression for computing the Fourier transform S(ω) of the photoacoustic signal s(t). Shown in Eq. 10:

H(ω)=S*(ω)e^{-iωτ} (10)

is an expression for computing the filter frequency response H(ω) based on the Fourier transform of the photoacoustic signal s(t), in which S*(ω) is the complex conjugate of S(ω). It can be observed that the filter frequency response of Eq. 10 requires the frequency character of the photoacoustic signal be known beforehand to determine the frequency response of the filter. In some embodiments, as shown by Eq. 11:

$$B ( t ) = {\int }_{- ∞}^{∞} r ( {t}^{′} ) s ( t + {t}^{′} ) {d}^{′} \qquad ( 11 )$$

the known modulation signal r(t) may be used for generating a cross-correlation with the photoacoustic signal. The cross-correlation output B(t) of Eq. 11 is expected to exhibit a peak at a time t equal to the acoustic signal travel time τ. Assuming that the temperature response and resulting acoustic response follow the illumination modulation (e.g., are coherent), Eq. 11 may allow calculation of the time delay, depth information, or both.

In some embodiments, the heterodyne mixing technique may be used to determine the travel time delay of the FD-PA signal. The FD-PA signal, as described above, may have similar frequency character as the modulation signal (e.g., coherence), albeit shifted in time due to the travel time of the acoustic signal. For example, a chirp modulation signal, such as r(t) of Eq. 7, may be used to modulate a CW light source. Heterodyne mixing uses the trigonometric identity of the following Eq. 12:

$$cos ( A ) cos ( B ) = \frac{1}{2} [ cos ( A - B ) - cos ( A + B ) ] \qquad ( 12 )$$

which shows that two signals may be combined by multiplication to give periodic signals at two distinct frequencies (i.e., the sum and the difference of the original frequencies). If the result is passed through a low-pass filter to remove the higher frequency term (i.e., the sum), the resulting filtered, frequency shifted signal may be analyzed. For example, Eq. 13 shows a heterodyne signal L(t):

$$L ( t ) = 〈 r ( t ) s ( t ) 〉 ≅ 〈 Kr ( t ) r ( t - \frac{R}{{c}_{a}} ) 〉 = \frac{1}{2} K cos ( \frac{R}{{c}_{a}} bt + θ ) \qquad ( 13 )$$

calculated by low-pass filtering (shown by angle brackets) the product of modulation signal r(t) and photoacoustic signal s(t). If the photoacoustic signal is assumed to be equivalent to the modulation signal, with a time lag R/cₐ due to travel time of the acoustic wave and amplitude scaling K, then a convenient approximation of Eq. 13 may be made, giving the rightmost expression of Eq. 13. Analysis of the rightmost expression of Eq. 13 may provide depth information, travel time, or both. For example, a fast Fourier transform (FFT) may be performed on the heterodyne signal, and the frequency associated with the highest peak may be considered equivalent to time lag Rb/cₐ. Assuming that the frequency scan rate b and the speed of sound cₐ are known, the depth R may be estimated.

The heterodyne mixing technique, the correlation technique, and other suitable processing techniques may be used to generate a time-domain signal using a FD-PA technique. In some embodiments, the processed FD-PA signal may be similar to that which would be received at the detector using TD-PA. The FD-PA technique may be desirable in some embodiments because of equipment requirements, processing requirements, power requirements, signal-to-noise requirements, any other suitable requirements, or any combination thereof. For example, a continuous wave laser diode may be smaller and require less power than a pulsed laser. It will be understood that in both TD-PA and FD-PA, the signal received at the detector is a time series of amplitude values. In some embodiments, elements within the time series (i.e., the time delays) received in the TD-PA technique may be considered to correlate to structures at a particular depth. Elements within the time series received in the FD-PA technique may require processing (e.g., heterodyne mixing) to correlate signal elements with structural elements.

In some embodiments, time domain processing steps may be carried out on a TD-PA signal, an FD-PA signal that has been processed into a form similar to a TD-PA signal using one of the aforementioned techniques, on any other suitable signal, or any combination thereof. For example, a time domain processing step may include isolating a structural element at a particular depth in a subject.

In some embodiments, frequency domain processing steps may be carried out on an FD-PA signal, a TD-PA signal, on any other suitable signal, or any combination thereof. In some embodiments, a time domain signal may be transformed to the frequency domain, e.g., by Fourier transform. For example, a frequency domain processing step may include determining a spectral ratio.

In some embodiments, a signal may be processed in the frequency domain, the time domain, any other suitable domain, or any combination thereof. An element (e.g., a peak corresponding to a particular depth in the subject) in a time domain signal may be isolated. The information in the isolated segment may be transformed to generate a frequency domain signal, using for example, a Fourier or other suitable transform. The frequency domain signal of the isolated peak may be processed, for example, to determine an absorption coefficient.

In some embodiments, a photoacoustic system may determine cardiac output and derivative physiological parameters. Cardiac output, as used herein, is understood to include the amount of blood pumped by the heart over a particular time interval. One clinical method of determining cardiac output includes rapidly injecting an amount of indicator (e.g., saline) into a blood vessel and subsequently measuring the time-dependent change in blood concentration (i.e., indicator dilution curve) downstream from the indicator injection site. A relatively fast return from the diluted state to a baseline blood concentration may indicate a high cardiac output, while a slow return may indicate low cardiac output. A photoacoustic system may be used to monitor the indicator dilution response and determine physiological parameters such as cardiac output.

In some embodiments, the optical absorption coefficient μₐ may be determined using the relative changes in amplitude within a frequency domain (FD-PA) photoacoustic signal. An acoustic signal S(ω), as described above, may be determined from a frequency-domain measurement, from the Fourier transform of a time-domain measurement, by any other suitable technique, or any combination thereof. For example, Eq. 14 shows an acoustic signal S(ω):

S(ω)=φ(λ₁)O(ω)H(ω)a(ω) (14)

where φ(λ₁) is the optical fluence at wavelength λ₁, O(ω) is the object spectrum and may be dependent upon extrinsic properties of the measurement (e.g., size of target, shape of target), H(ω) is the system dependent response and may be dependent upon intrinsic properties of the photoacoustic system, and a(ω) is the acoustic attenuation effect and may be dependent upon the intrinsic properties of the target. In some embodiments, a target may be exposed to photonic signals of more than one wavelength. The system dependent response H(ω) and intrinsic target attenuation effect a(ω) may not change with changes in the exposing wavelength. Two signals may be compared by dividing them. For example, Eq. 15 shows the comparison of signals S₁(ω) and S₂(ω):

$$\frac{{S}_{1} ( ω ) = φ ( {λ}_{1} ) {O}_{1} ( ω ) H ( ω ) a ( ω )}{{S}_{2} ( ω ) = φ ( {λ}_{2} ) {O}_{2} ( ω ) H ( ω ) a ( ω )} \qquad ( 15 )$$

which may be reduced to Eq. 16 by arithmetic steps:

$$\frac{{S}_{1} ( ω ) = φ ( {λ}_{1} ) {O}_{1} ( ω )}{{S}_{2} ( ω ) = φ ( {λ}_{2} ) {O}_{2} ( ω )} \qquad ( 16 )$$

Object spectrum O(t) in units of time may be defined as shown in Eq. 17:

O(t)=Γμₐe^{-μ}_{^{a}}^{ct} (17)

Taking the Fourier transform of Eq. 17 and combining it with Eq. 16 may result in, for example, Eq. 18:

$$\frac{{S}_{1} ( ω )}{{S}_{2} ( ω )} = \frac{φ ( {λ}_{1} ) \sqrt{{( \frac{ω}{{μ}_{{a}_{1}}} )}^{2} + {c}^{2}}}{φ ( {λ}_{2} ) \sqrt{{( \frac{ω}{{μ}_{{a}_{2}}} )}^{2} + {c}^{2}}} \qquad ( 18 )$$

The ratio S₁(ω)/S₂(ω) as shown in Eq. 18 may be fitted to the data to extract values for absorption coefficients μₐ_{₁}, absorption coefficient μhd a_{_{2, and fluence ratio φ(λ}}₁)/φ(λ₂) for a given pair of photonic signals at wavelengths λ₁ and λ₂.

As shown by Eqs. 15-18, the absolute value of the absorption coefficient may be determined without a secondary measurement (e.g., oblique-incidence diffuse reflectance).

In some embodiments, the absorptivity of a blood parameter may be known at one or more particular wavelengths of light. An absorption coefficient μ_{a,λ}_{₁} may be given by the following:

μ_{a,λ}_{₁}=c·ε_{λ}_{₁} (19)

where ε_{λ}_{₁} (presumed known) is the absorptivity of a particular blood solute at wavelength λ₁ and c is the concentration of the solute. In some embodiments, a second light source of a second wavelength λ₂, different from the first, may be used to determine blood oxygen saturation. In some embodiments, photoacoustic signals at particular wavelengths may be extracted from photoacoustic data related to a frequency modulated photonic signal, where the frequency modulation is a chirp signal. Where a first and a second absorption coefficient have been determined (e.g., using Eq. 15-18) at a first and second wavelength, the absorption coefficients μ_{a,λ}_{₁} and μ_{a0λ}_{₂} may be represented by the following:

μ_{a,λ}_{₁}=ε_{ox,λ}_{₁}cₒₓ+ε_{deox,λ}_{₁}c_{deox} (20)

μ_{a,λ}_{₂}=ε_{ox,λ}₂cₒₓ+ε_{deox,λ}_{₂}c_{deox} (21)

where ε_{ox,λ}_{₁} is the absorptivity of oxyhemoglobin at λ₁, ε_{deox,λ}_{₁} is the absorptivity of deoxyhemoglobin at λ₁, ε_{ox,λ}_{₂} is the absorptivity of oxyhemoglobin at λ₂, ε_{deox,λ}_{₂} is the absorptivity of deoxyhemoglobin at λ₂, cₒₓ is the concentration of oxyhemoglobin, and c_{deox} is the concentration of deoxyhemoglobin. These two equations contain two unknowns (i.e., cₒₓ and c_{deox}) and therefore may be solved to determine the concentration of oxyhemoglobin and the concentration of deoxyhemoglobin.
The total hemoglobin may be determined by:

tHb=cₒₓ+c_{deox} (22)

Additionally, blood oxygen saturation S_{O2} may be determined by the following:

$${SO}_{2} = \frac{{c}_{ox}}{{c}_{ox} + {c}_{deox}} \qquad ( 23 )$$

which may, for example, be an arterial blood oxygen saturation or venous oxygen saturation depending upon the type of blood vessel. It will be understood that Eqs. 19-23 provide illustrative examples of formulas used to determine physiological parameters from photoacoustic measurements. Any suitable equations, models, other suitable mathematical construct, look-up table, database, or other reference may be used to determine one or more physiological parameters. For example, in some embodiments, physiological parameters may be tabulated for discrete values of absorption coefficient at one or more wavelengths.

It will be understood that while some of the equations referenced herein are continuous functions, the processing equipment may be configured to use digital or discrete forms of the equations in processing the acquired photoacoustic signals. It will be understood that Fourier transforms may include Fourier transforms, inverse Fourier transforms, fast Fourier transforms, discrete Fourier transforms, chirplet transforms, any other suitable time frequency analysis, or any combination thereof.

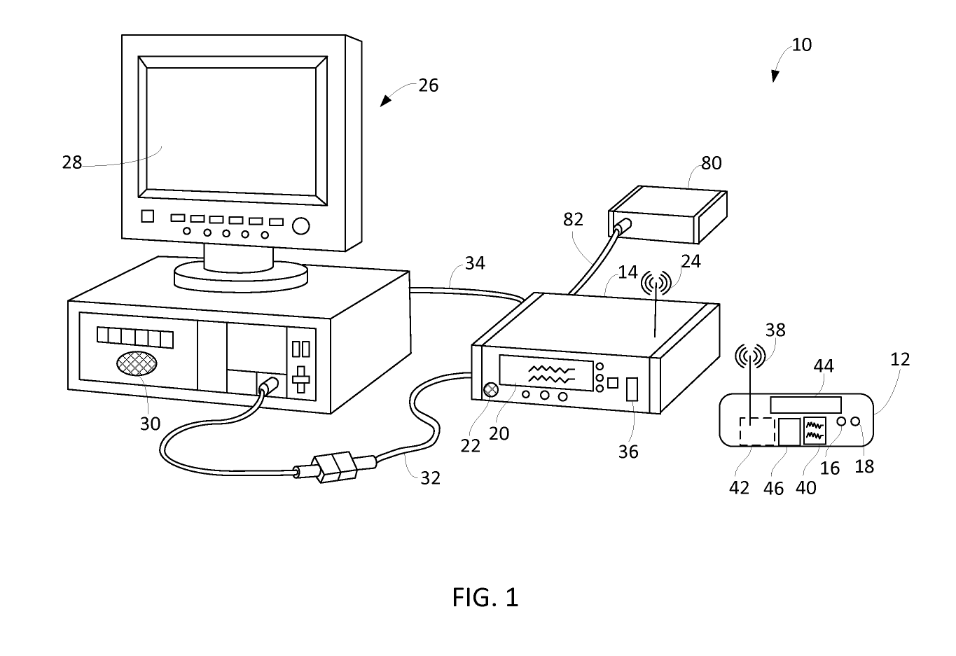

The following description and accompanying FIGS. 1-11 provide additional details and features of determining absorption coefficients used to determine physiological parameters.

FIG. 1 shows an illustrative physiological monitoring system in accordance with some embodiments of the present disclosure. System 10 may include sensor unit 12 and monitor 14. In some embodiments, sensor unit 12 may be part of a photoacoustic monitor or imaging system. Sensor unit 12 may include a light source 16 for emitting light at one or more wavelengths into a subject's tissue, which may but need not correspond to visible light, into a subject's tissue. Light source 16 may provide a photonic signal including any suitable electromagnetic radiation such as, for example, a radio wave, a microwave wave, an infrared wave, a visible light wave, ultraviolet wave, any other suitable light wave, or any combination thereof. A detector 18 may also be provided in sensor unit 12 for detecting the acoustic (e.g., ultrasound) response that travels through the subject's tissue. Any suitable physical configuration of light source 16 and detector 18 may be used. In some embodiments, sensor unit 12 may include multiple light sources and/or acoustic detectors, which may be spaced apart.

System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12 (e.g., a photoplethysmograph sensor). Multiple sensor units may be capable of being positioned at two different locations on a subject's body.

In some embodiments, system 10 may include two or more sensors forming a sensor array in lieu of either or both of the sensor units. In some embodiments, a sensor array may include multiple light sources, detectors, or both. It will be understood that any type of sensor, including any type of physiological sensor, may be used in one or more sensor units in accordance with the systems and techniques disclosed herein. It will be understood that any number of sensors measuring any number of physiological signals may be used to determine physiological information in accordance with the techniques described herein.

In some embodiments, the sensor may be wirelessly connected to monitor 14 (e.g., via wireless transceivers 38 and 24) and include its own battery or similar power source 44. In some embodiments, sensor unit 12 may draw its power from monitor 14 and be communicate with monitor 14 via a physical connection such as a wired connection (not shown). Sensor unit 12, monitor 14, or both, may be configured to calculate physiological parameters based at least in part on data relating to light emission and acoustic detection received at one or more sensor units such as sensor unit 12. For example, sensor unit 12, monitor 14, or both, may be configured to determine blood oxygen saturation (e.g., arterial, venous, or both), pulse rate, hemoglobin concentration (e.g., oxygenated, deoxygenated, or total), any other suitable physiological parameters, or any combination thereof. In some embodiments, some or all calculations may be performed on sensor unit 12 (i.e., using processing equipment 42) or an intermediate device and the result of the calculations may be passed to monitor 14. Further, monitor 14 may include monitor display 20 configured to display the physiological parameters or other information about the system. Sensor unit 12 may also include a sensor display 40 configured to display the physiological parameters or other information about the system and a user interface 46. In an exemplary embodiment, processing equipment 42 may be configured to operate light source 16 and detector 18 to generate and process acoustic signals, communicate with display sensor 40 to display values such as signal quality and power levels, receive signals from user input 46, and control wireless transceiver 38 to communicate data (e.g., acoustic output signals) with monitor 14.

In the embodiment shown, monitor 14 may also include speaker 22 to provide an audible sound that may be used in various other embodiments, such as for example, sounding an audible alarm in the event that a subject's physiological parameters are not within a predefined normal range. In another embodiment, sensor unit 12 may communicate such information to the user, e.g., using sensor display 40, an audible source such as a speaker, vibration, tactile, or any other way for communicating a status to a user, such as for example, in the event that a subject's physiological parameters are not within a predefined normal range.

In some embodiments, sensor unit 12 may be communicatively coupled to monitor 14 via a wireless system, utilizing antenna 38 of sensor unit 12 and antenna 24 of monitor 14. Antenna 38 may be external or internal to sensor unit 12, and capable of transmitting signals, receiving signals, or both transmitting and receiving signals, via amplitude modulated RF, frequency modulated RF, Bluetooth, IEEE 802.11, WiFi, WiMax, cable, satellite, infrared, any other suitable transmission scheme, or any combination thereof. Communication between the sensor unit 12 and monitor 14 may also be carried over a cable (not shown) to an input 36 of monitor 14, or to a multi-parameter physiological monitor 26 (described below). The cable may include electronic conductors (e.g., wires for transmitting electronic signals from detector 18, or a partially or fully processed signal from sensor unit 12), optical fibers (e.g., multi-mode or single-mode fibers for transmitting emitted light from light source 16), any other suitable components, any suitable insulation or sheathing, or any combination thereof. Monitor 14 may include a sensor interface configured to receive physiological signals from sensor unit 12, provide signals and power to sensor unit 12, transfer data specific to the subject, general to the physiological parameter being measured, or both, or otherwise communicate with sensor unit 12. The sensor interface may include any suitable hardware, software, or both, which may allow communication between monitor 14 and sensor unit 12.

In the illustrated embodiment, system 10 includes multi-parameter physiological monitor 26. The monitor 26 may include a cathode ray tube display, a flat panel display (as shown) such as a liquid crystal display (LCD) or a plasma display, or may include any other type of monitor now known or later developed. Multi-parameter physiological monitor 26 may be configured to calculate physiological parameters and to provide a multi-parameter physiological monitor display 28 for information from sensor unit 12, monitor 14, or both, and from other medical monitoring devices or systems (not shown). For example, multi-parameter physiological monitor 26 may be configured to display an estimate of, for example, physiological parameters such as pulse rate, hemoglobin concentration (e.g., oxygenated, deoxygenated, total, or a combination thereof), blood oxygen saturation (e.g., arterial, venous, or both), temperature, cardiac output (CO), intrathoracic blood volume (ITBV), intrathoracic circulatory volume (ITCV), global end-diastolic volume (GEDV), pulmonary circulatory volume (PCV), extravascular lung water (EVLW), any other suitable hemodynamic parameters, any other suitable physiological parameters, any physiological modulations thereof, or any combination thereof, generated by sensor unit 12 or monitor 14. Multi-parameter physiological monitor 26 may include a speaker 30.

Monitor 14 may be communicatively coupled to multi-parameter physiological monitor 26 via a cable 32 or 34 that is coupled to a sensor input port or a digital communications port, respectively and/or may communicate wirelessly (not shown). The multi-parameter physiological monitor 26 may also be communicatively coupled to sensor unit 12 with or without the presence of monitor 14. Sensor unit 12 may be coupled to the multi-parameter physiological monitor 26 by a wireless connection using wireless transceiver 38 and a transceiver (not shown) on multi-parameter physiological monitor 26, or by a cable (not shown). In addition, sensor unit 12, monitor 14, or multi-parameter physiological monitor 26 may be coupled to a network to enable the sharing of information with servers or other workstations (not shown). In some embodiments this network may be a local area network, which may be further coupled through the internet or other wide area network for remote monitoring. Sensor unit 12, monitor 14 and multi-parameter physiological monitor 26 may be powered by a battery (not shown) or by a conventional power source such as a wall outlet.

Calibration device 80, which may be powered by monitor 14, a battery, or by a conventional power source such as a wall outlet, may include any suitable calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

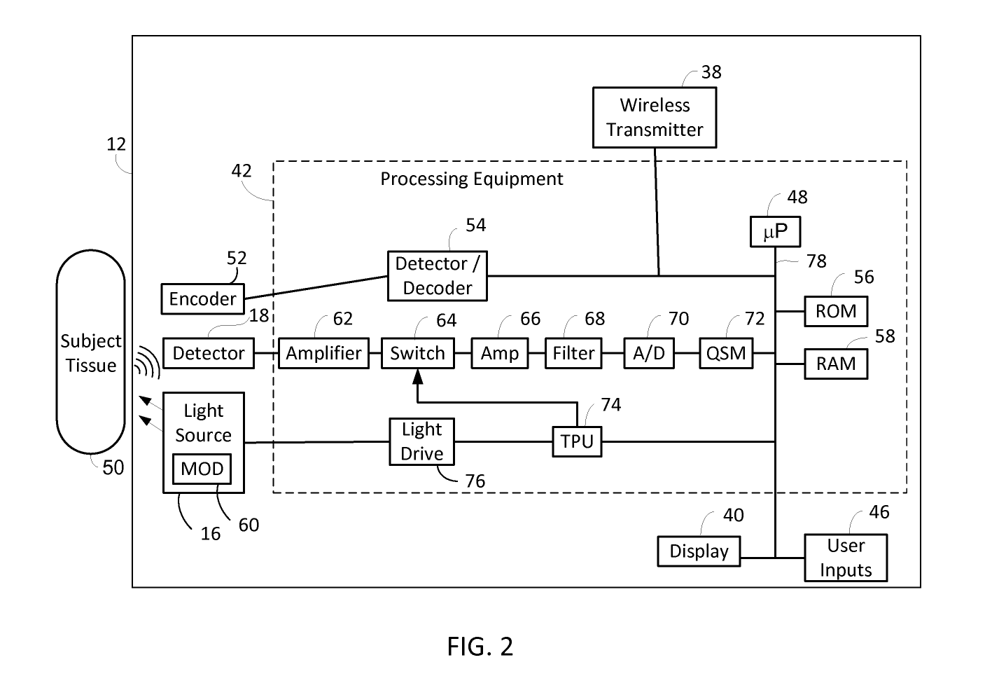

FIG. 2 is a block diagram of an illustrative sensor unit 12 of physiological monitoring system 10 of FIG. 1, which may be coupled to a subject 50 in accordance with some embodiments of the present disclosure. It will be understood that processing equipment 42 of FIG. 2 may be included fully or partially included in sensor unit 12, fully or partially in monitor 14 of FIG. 1, fully or partially in multi-parameter physiological monitor 26 of FIG. 1, in any other suitable arrangement, or any combination thereof. It will be understood that any displayed information may be displayed on sensor display 40, monitor display 20 of FIG. 1, multi-parameter physiological monitor display 28 of FIG. 1, any other suitable display, or any combination thereof.

Sensor unit 12 may include light source 16, detector 18, and encoder 52. In some embodiments, light source 16 may be configured to emit one or more wavelengths of light (e.g., visible, infrared) into a subject's tissue 50. Hence, light source 16 may provide red light, IR light, any other suitable light, or any combination thereof, that may be used to calculate the subject's physiological parameters. In some embodiments, the red wavelength may be between about 600 nm and about 700 nm, and the IR wavelength may be between about 800 nm and about 1000 nm. In embodiments where a sensor array is used in place of a single sensor, each sensor may be configured to provide light of a single wavelength. For example, a first sensor may emit only a Red light while a second may emit only an IR light. In a further example, the wavelengths of light used may be selected based on the specific location of the sensor. In some embodiments, light source 16 may include one or more continuous wave laser diodes. Light source 16 may include optical elements such as mirrors, prisms, diffusers, other suitable elements, or any combination thereof.

It will be understood that, as used herein, the term “light” may refer to energy produced by electromagnetic radiation sources. Light may be of any suitable wavelength and intensity, and modulations thereof, in any suitable shape and direction. Detector 18 may be chosen to be specifically sensitive to the acoustic response of the subject's tissue arising from use of light source 16. It will also be understood that, as used herein, the “acoustic response” shall refer to pressure and changes thereof caused by a thermal response (e.g., expansion and contraction) of tissue to light absorption by the tissue or constituent thereof.

In some embodiments, detector 18 may be configured to detect the acoustic response of tissue to the photonic excitation caused by the light source. In some embodiments, detector 18 may be a piezoelectric transducer which may detect force and pressure and output an electrical signal via the piezoelectric effect. In some embodiments, detector 18 may be a Faby-Pérot interferometer, or etalon. For example, a thin film (e.g., composed of a polymer) may be irradiated with reference light, which may be internally reflected by the film. Pressure fluctuations may modulate the film thickness, thus causing changes in the reference light reflection which may be measured and correlated with the acoustic pressure. In some embodiments, detector 18 may be configured or otherwise tuned to detect acoustic response in a particular frequency range. Detector 18 may convert the acoustic pressure signal into an electrical signal (e.g., using a piezoelectric material, photodetector of a Faby-Pérot interferometer, or other suitable device). After converting the received acoustic pressure signal to an electrical optical, and/or wireless signal, detector 18 may send the signal to processing equipment 42, where physiological parameters may be calculated based on the photoacoustic activity within the subject's tissue 50. The signal outputted from detector 18 and/or a pre-processed signal derived thereof, will be referred to herein as a photoacoustic signal.

In some embodiments, encoder 52 may contain information about sensor unit 12, such as what type of sensor it is (e.g., where the sensor is intended to be placed on a subject), the wavelength(s) of light emitted by light source 16, the intensity of light emitted by light source 16 (e.g., output wattage or Joules), the mode of light source 16 (e.g., pulsed versus CW), any other suitable information, or any combination thereof. This information may be used by processing equipment 42 to select appropriate algorithms, lookup tables, and/or calibration coefficients stored in processing equipment 42 for calculating the subject's physiological parameters.

Encoder 52 may contain information specific to subject's tissue 50, such as, for example, the subject's age, weight, and diagnosis. This information about a subject's characteristics may allow processing equipment 42 to determine, for example, subject-specific threshold ranges in which the subject's physiological parameter measurements should fall and to enable or disable additional physiological parameter algorithms. Encoder 52 may, for instance, be a coded resistor that stores values corresponding to the type of sensor unit 12 or the type of each sensor in the sensor array, the wavelengths of light emitted by light source 16 on each sensor of the sensor array, and/or the subject's characteristics. In some embodiments, encoder 52 may include a memory on which one or more of the following information may be stored for communication to processing equipment 42: the type of the sensor unit 12; the wavelengths of light emitted by light source 16; the particular acoustic range that each sensor in the sensor array is monitoring; the particular acoustic spectral characteristics of a detector; a signal threshold for each sensor in the sensor array; any other suitable information; or any combination thereof.

In some embodiments, signals from detector 18 and encoder 52 may be transmitted to processing equipment 42. In the embodiment shown, processing equipment 42 may include a general-purpose microprocessor 48 connected to an internal bus 78. Microprocessor 48 may be adapted to execute software, which may include an operating system and one or more applications, as part of performing the functions described herein. Also connected to bus 78 may be a read-only memory (ROM) 56, a random access memory (RAM) 58, user inputs 46, sensor display 40, and speaker 22 of FIG. 1.

RAM 58 and ROM 56 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules, or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, time processing unit (TPU) 74 may provide timing control signals to light drive circuitry 76, which may control the activation of light source 16. For example, TPU 74 may control pulse timing (e.g., pulse duration and inter-pulse interval) for TD-PA monitoring system. TPU 74 may also control the gating-in of signals from detector 18 through amplifier 62 and switching circuit 64. The received signal from detector 18 may be passed through amplifier 66, low pass filter 68, and analog-to-digital converter 70. The digital data may then be stored in a queued serial module (QSM) 72 (or buffer) for later downloading to RAM 58 as QSM 72 is filled. In some embodiments, there may be multiple separate parallel paths having components equivalent to amplifier 62, filter 68, and/or analog-to-digital converter 70 for multiple light wavelengths or spectra received. Any suitable combination of components (e.g., microprocessor 48, RAM 58, analog-to-digital converter 70, any other suitable component shown or not shown in FIG. 2) coupled by bus 78 or otherwise coupled (e.g., via an external bus), may be referred to as “processing equipment.”

In the embodiment shown, light source 16 may include modulator 60, in order to, for example, control the intensity of light source 16. Modulator 60 may include any suitable optics including choppers, other suitable equipment to modulate a signal, or any combination thereof. Modulator 60 may be configured to provide intensity modulation, spatial modulation, time modulation, any other suitable optical signal modulations, or any combination thereof. For example, light source 16 may be a CW light source, and modulator 60 may provide intensity modulation of the CW light source such as using a linear sweep modulation. In some embodiments, modulator 60 may be included in light drive 60, or other suitable components of physiological monitoring system 10, or any combination thereof.

In some embodiments, microprocessor 48 may determine the subject's physiological parameters such as pulse rate, hemoglobin concentration (e.g., oxygenated, deoxygenated, total, or a combination thereof), blood oxygen saturation (e.g., arterial, venous, or both), temperature, cardiac output (CO), intrathoracic blood volume (ITBV), intrathoracic circulatory volume (ITCV), global end-diastolic volume (GEDV), pulmonary circulatory volume (PCV), extravascular lung water (EVLW), any other suitable hemodynamic parameters, any other suitable physiological parameters, any physiological modulations thereof, or any combination thereof, using various algorithms and/or look-up tables based on the value of the received signals and/or data corresponding to the acoustic response received by detector 18. Signals corresponding to information about subject 50, and particularly about the acoustic signals emanating from a subject's tissue over time, may be transmitted from encoder 52 to decoder 54. These signals may include, for example, encoded information relating to subject characteristics. Decoder 54 may translate these signals to enable the microprocessor to determine the thresholds based at least in part on algorithms or look-up tables stored in ROM 56. In some embodiments, user inputs 46 may be used enter information, select one or more options, provide a response, input settings, any other suitable inputting function, or any combination thereof. User inputs 46 may be used to enter information about the subject, such as, for example, age, weight, height, diagnosis, medications, treatments, and so forth. In some embodiments, sensor display 40 may exhibit a list of values, which may generally apply to the subject, such as, for example, age ranges or medication families, which the user may select using user inputs 46.

The acoustic signal attenuated by the tissue of subject 50 can be degraded by noise, among other sources. Movement of the subject may also introduce noise and affect the signal. For example, the contact between the detector and the skin, or the light source and the skin, can be temporarily disrupted when movement causes either to move away from the skin. Another potential source of noise is electromagnetic coupling from other electronic instruments.

Noise (e.g., from subject movement) can degrade a sensor signal relied upon by a care provider, without the care provider's awareness. This is especially true if the monitoring of the subject is remote, the motion is too small to be observed, or the care provider is watching the instrument or other parts of the subject, and not the sensor site. Processing sensor signals may involve operations that reduce the amount of noise present in the signals, control the amount of noise present in the signal, or otherwise identify noise components in order to prevent them from affecting measurements of physiological parameters derived from the sensor signals.

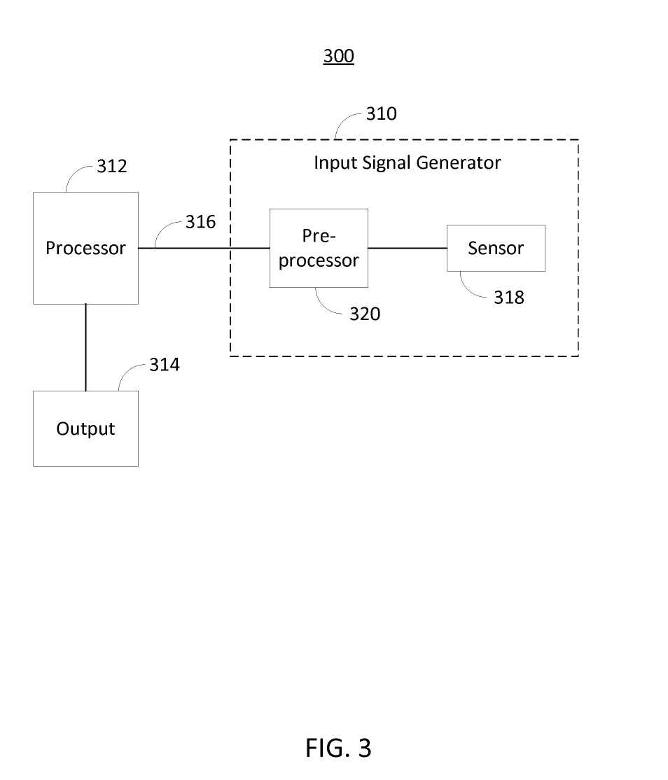

FIG. 3 is a block diagram of an illustrative signal processing system in accordance with some embodiments of the present disclosure. Signal processing system 300 may implement the signal processing techniques described herein. In some embodiments, signal processing system 300 may be included in a physiological monitoring system (e.g., physiological monitoring system 10 of FIG. 1 or sensor unit 12 of FIG. 1-2). In the illustrated embodiment, input signal generator 310 generates an input signal 316. As illustrated, input signal generator 310 may include pre-processor 320 coupled to sensor 318, which may provide input signal 316. In some embodiments, pre-processor 320 may be a photoacoustic module and input signal 316 may be a photoacoustic signal. In some embodiments, pre-processor 320 may be any suitable signal processing device and input signal 316 may include one or more photoacoustic signals and one or more other physiological signals, such as a photoplethysmograph signal. It will be understood that input signal generator 310 may include any suitable signal source, signal generating data, signal generating equipment, or any combination thereof to produce input signal 316. Input signal 316 may be a single signal, or may be multiple signals transmitted over a single pathway or multiple pathways.

Pre-processor 320 may apply one or more signal processing operations to the signal generated by sensor 318. For example, pre-processor 320 may apply a pre-determined set of processing operations to the signal provided by sensor 318 to produce input signal 316 that can be appropriately interpreted by processor 312, such as performing A/D conversion. In some embodiments, A/D conversion may be performed by processor 312. Pre-processor 320 may also perform any of the following operations on the signal provided by sensor 318: reshaping the signal for transmission, multiplexing the signal, modulating the signal onto carrier signals, compressing the signal, encoding the signal, and filtering the signal.

In some embodiments, input signal 316 may be coupled to processor 312. Processor 312 may be any suitable software, firmware, hardware, or combination thereof for processing input signal 316. For example, processor 312 may include one or more hardware processors (e.g., integrated circuits), one or more software modules, and computer-readable media such as memory, firmware, or any combination thereof. Processor 312 may, for example, be a computer or may be one or more chips (i.e., integrated circuits). Processor 312 may, for example, include an assembly of analog electronic components. Processor 312 may calculate physiological information. For example, processor 312 may perform time domain calculations, spectral domain calculations, time-spectral transformations (e.g., fast Fourier transforms, inverse fast Fourier transforms), any other suitable calculations, or any combination thereof. Processor 312 may perform any suitable signal processing of input signal 316 to filter input signal 316, such as any suitable band-pass filtering, adaptive filtering, closed-loop filtering, any other suitable filtering, and/or any combination thereof. Processor 312 may also receive input signals from additional sources (not shown). For example, processor 312 may receive an input signal containing information about treatments provided to the subject. Additional input signals may be used by processor 312 in any of the calculations or operations it performs in accordance with processing system 300.

In some embodiments, all or some of pre-processor 320, processor 312, or both, may be referred to collectively as processing equipment. For example, processing equipment may be configured to amplify, filter, sample and digitize input signal 316 (e.g., using an analog to digital converter), and calculate physiological information from the digitized signal.

Processor 312 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. In some embodiments, processor 312 may store physiological measurements or previously received data from signal 316 in a memory device for later retrieval. In some embodiments, processor 312 may store calculated values, such as blood oxygen saturation (e.g., arterial, venous, or both), hemoglobin concentration (e.g., oxygenated, deoxygenated, or total), cardiac output, temperature, any other suitable calculated values, or combinations thereof, in a memory device for later retrieval.

Processor 312 may be coupled to output 314. Output 314 may be any suitable output device such as one or more medical devices (e.g., a medical monitor that displays various physiological parameters, a medical alarm, or any other suitable medical device that either displays physiological parameters or uses the output of processor 312 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

It will be understood that system 300 may be incorporated into system 10 (FIG. 1), in which, for example, input signal generator 310 may be implemented as part of sensor unit 12 (FIGS. 1 and 2), monitor 14 (FIG. 1), and processing equipment 42 (FIG. 2), and processor 312 may be implemented as part of monitor 14 (FIG. 1) and processing equipment 42 (FIG. 2). In some embodiments, portions of system 300 may be configured to be portable. For example, all or part of system 300 may be embedded in a small, compact object carried with or attached to the subject (e.g., a watch, other piece of jewelry, or a smart phone). In some embodiments, a wireless transceiver (not shown) may also be included in system 300 to enable wireless communication with other components of system 10 (FIG. 1). As such, system 10 (FIG. 1) may be part of a fully portable and continuous physiological monitoring solution. In some embodiments, a wireless transceiver (not shown) may also be included in system 300 to enable wireless communication with other components of system 10. For example, pre-processor 320 may output signal 316 (e.g., which may be a pre-processed photoacoustic signal) over BLUETOOTH, IEEE 802.11, WiFi, WiMax, cable, satellite, Infrared, any other suitable transmission scheme, or any combination thereof. In some embodiments, a wireless transmission scheme may be used between any communicating components of system 300.

It will be understood that while some of the equations referenced herein are continuous functions, the processing equipment may be configured to use digital or discrete forms of the equations in processing the acquired photoacoustic signals. It will be understood that Fourier transforms may include Fourier transforms, inverse Fourier transforms, fast Fourier transforms, discrete Fourier transforms, chirplet transforms, any other suitable time frequency analysis, or any combination thereof.

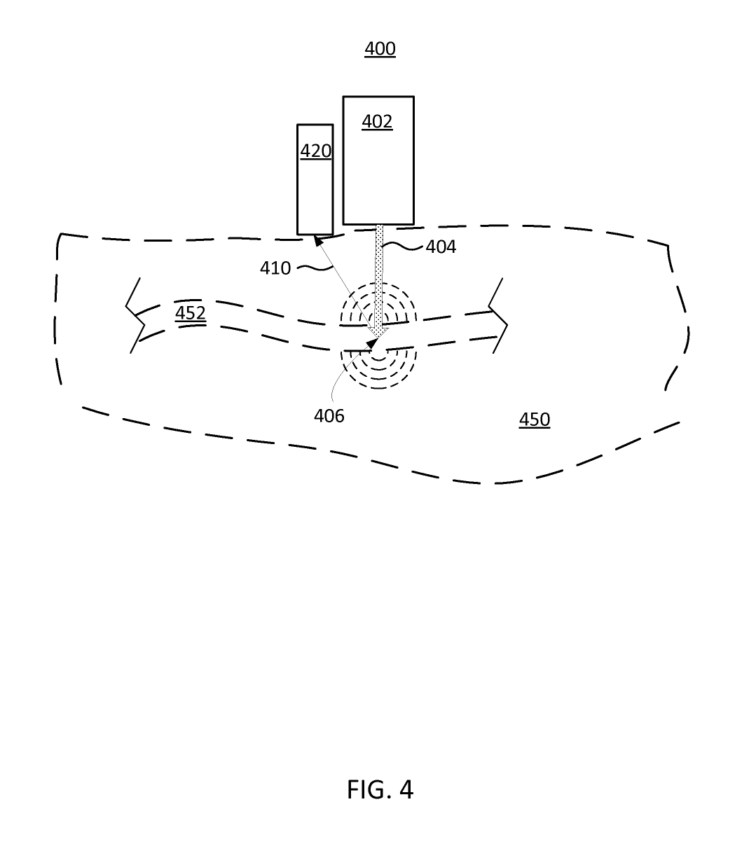

FIG. 4 is an illustrative photoacoustic arrangement in accordance with some embodiments of the present disclosure. The arrangement 400 may include light source 402, controlled by a suitable light drive (e.g., a light drive of system 300 of FIG. 3 or system 10 of FIG. 1, although not shown in FIG. 4). Light source 402 may provide photonic signal 404 to subject tissue 450 including blood vessel 452. Photonic signal 404 may be attenuated along its path length by subject tissue 450 prior to reaching target area 406 of blood vessel 452. It will be understood that photonic signal 404 may scatter in subject tissue 450 and need not travel in a well-formed beam as illustrated. It will also be understood that photonic signal 404 may generally travel through and beyond blood vessel 452. A constituent of the blood in blood vessel 452 such as, for example, hemoglobin, may absorb at least some of photonic signal 404. Accordingly, the blood may exhibit an acoustic pressure response via the photoacoustic effect, which may act on the surrounding tissues of blood vessel 452. Photoacoustic signal 410 may be generated near target area 406 of blood vessel 452 and may travel through subject 450 in all directions. Acoustic detector 420 (i.e., a photoacoustic detector) may detect acoustic pressure signals corresponding to photoacoustic signal 410. Acoustic detector 420 may output a signal for further processing.

It will be understood that the arrangement 400 is merely exemplary, and that in some embodiments one or more photonic signals may interact with one or more blood vessels within subject tissue 450. For example, photonic signal 404 may interact with several blood vessels, and each blood vessel may generate an acoustic pressure wave that may be detected by acoustic detector 420. Photonic signal 404 may also be modulated such that the one or more acoustic signals detected by acoustic detector 420 change over time. It will also be understood that light source 402 may include one or more emitters configured to emit light at one or more wavelengths. It will also be understood that the system may include one or more light source 402, one or more detector 420, or any combination thereof. The one or more light sources and one or more detectors may be located in the same location, in separate locations, in any other suitable arrangement, or any combination thereof. For example, the system may include two light sources and one detector located on the arm of a subject. In a further example, the system may include a light source and detector on the left arm of a subject, and a light source and detector on the left arm of a subject.

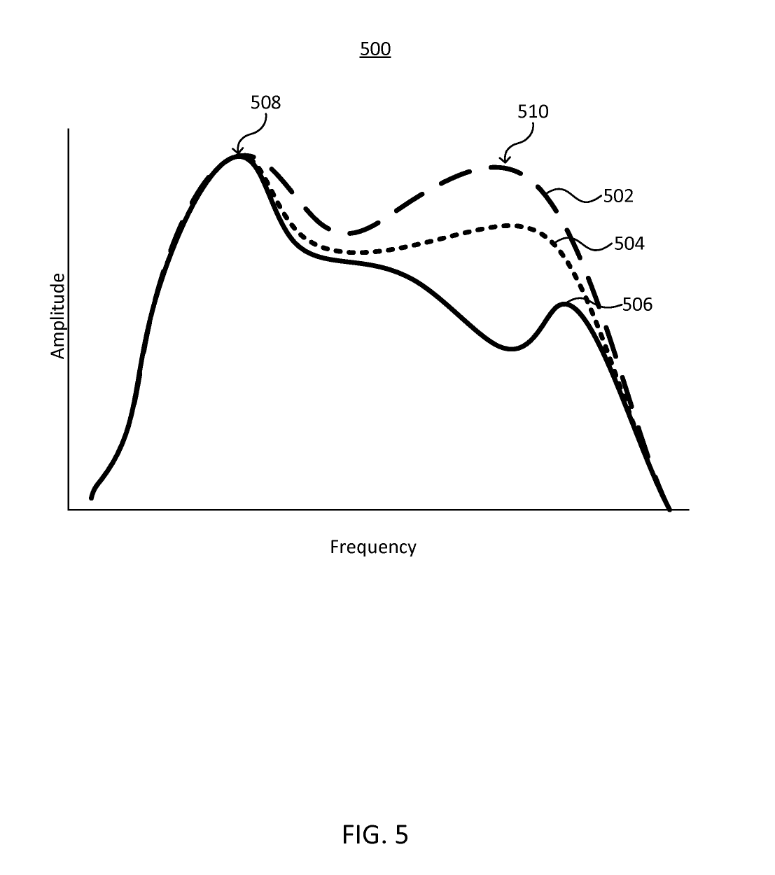

FIG. 5 is an illustrative plot 500 of frequency-domain photoacoustic signals in accordance with some embodiments of the present disclosure. The abscissa of plot 500 is presented in units of frequency, for example, in megahertz. The units of the ordinate of plot 500 may correspond to the amplitude of detected photoacoustic signals as a function of frequency. Plot 500 includes curve 502, curve 504, and curve 506. Each of curves 502-506 corresponds to a frequency-domain photoacoustic signal. For example, each curve may correspond to a photoacoustic signal generated by a particular component of tissue (e.g., a blood vessel) in response to a photonic signal of a different wavelength. As described above, the signal may be generated by processing of a signal received using FD-PA, TD-PA, any other suitable photoacoustic technique, or any combination thereof. In some embodiments, the frequency range of curve 502, curve 504, and curve 506 may depend on the frequency of the modulation of the light source. For example, a photoacoustic signal corresponding to a light source modulated in the megahertz range may be detected in that same megahertz range. In some embodiments, higher frequencies may be desired over lower frequencies for determining physiological parameters due to an increased sensitivity to absorption coefficient changes at higher frequencies. In some embodiments, the sampling rate of a digitizer may limit the frequency range of a frequency domain signal. For example, a FD-PA technique may use a digitizer sampling in the gigasamples per second range, prior to generating and processing a time domain signal by heterodyne mixing, and finally transforming that signal into a frequency domain signal such as the signals of plot 500.

In some embodiments, the system may include a time-domain photoacoustic signal. The time-domain photoacoustic signal may be received by the detector in a TD-PA technique, may be generated by processing of a FD-PA signal (e.g., heterodyne mixing), it may be generated by a transform of a frequency domain signal (e.g., Fourier transform), it may be determined or generated by any other suitable technique, or any combination thereof.

In some embodiments (e.g., frequency domain-photoacoustic spectroscopy), the received signals may be generated by varying a modulation frequency for a light source illuminating the subject, for example, a chirp modulation. A frequency-domain photoacoustic signal generated by varying a light source modulation frequency may be further processed using a correlation or heterodyne mixing technique. The system may generate a time domain signal using the correlation or heterodyne mixing technique.

In some embodiments, a time-domain photoacoustic signal may include peaks, valleys, and other elements related to particular structural elements of the subject. The time domain elements may, for example, correspond to depth in a subject. For example, a signal measured at the wrist may include a peak related to the skin and a peak related to the radial artery. The system may use only a portion of the signal, for example the photoacoustic peak related to the radial artery, for further processing. Thus, the system may identify and isolate structures at a particular depth in the subject by processing a time-domain signal. In some embodiments, this isolated time-domain signal may be transformed to a frequency domain signal using, for example, a Fourier transform.

In some embodiments, photoacoustic imaging may be used to identify particular structural elements of the subject. For example, photoacoustic imaging may provide a 2-dimensional image of structural elements beneath the skin of a subject. Data from the photoacoustic image may be used in part to isolate a photoacoustic signal relating to a particular structural element of the subject. Thus, the system may use 2-dimensional photoacoustic imaging to identify the position of structures in relation to the surface of a subject. It will be understood that this 2-dimensional alignment may be used in combination with the aforementioned time domain processing technique to isolate a time domain element.

In some embodiments, the amplitudes of the curves in plot 500 may be normalized. For example, an additional reference signal may be used for normalization. The reference may be generated using a calibration tool such as calibration device 80 of FIG. 1. In some embodiments, the system may use a reference sample with known optical properties for calibration. In some embodiments, the system may normalize a sample using peaks in the generated spectrum. It will be understood that these normalization techniques are merely exemplary and that any suitable technique or combination of techniques may be employed. It will also be understood that the signals may not be normalized.

In some embodiments, the frequency range of the plot may be limited by the detector and/or other components of the system. For example, an ultrasound detector may have a bandwidth of 50-80% of its central frequency at a 6 dB rolloff. Frequencies beyond the limit may be highly attenuated and not useful for processing. In some embodiments, corrections related to the frequency response of the sensor may be included in a normalization step.

In some embodiments, curve 502, curve 504, and curve 506 may contain peaks at similar frequencies but the amplitudes may differ. For example, at frequency 510, curve 502 indicates a larger amplitude than curve 504, which indicates a larger amplitude than curve 506. In some embodiments, the curves may indicate similar amplitudes at low frequencies and different amplitudes at higher frequencies. For example, at frequency 508, curve 502, curve 504, and curve 506 may display substantially similar amplitudes. In some embodiments, the shape of the curves may be related to the absorption coefficient of the illuminated target area (e.g., the structural component or components included in the signal). In some embodiments, the shape of curve 502, curve 504, and curve 506 may be related to a structural element, as described above. In some embodiments, where the structural element is related to blood, the absorption coefficient, and thus the shape of the curve, may be related to the hemoglobin concentration. For example, the relatively higher amplitude curve 502 at frequency 510 may indicate a high hemoglobin concentration. Similarly, the relatively low amplitude of curve 506 at frequency 510 may indicate a low hemoglobin concentration.

In some embodiments, the relative amplitudes may be used to determine an absorption coefficient. For example, two curves may be divided and the ratio fit to Eq. 18 to extract μₐ. In some embodiments, the extracted μₐ may be independent of optical fluence and acoustic signal attenuation. In some embodiments, the curves of Plot 500 may correspond to different levels of hemoglobin (or different structures) measured using the same wavelength of light. Thus, the fluence ratio in Eq. 18 may be equal to 1, and the information from two photoacoustic signals can be used to extract the two absorption coefficients. In some embodiments, two wavelengths of light may be used to measure the same structure. Thus, two photoacoustic signals may be used to extract the fluence ratio and the one absorption coefficient. It will be understood that the system may use more complex combinations of structures and wavelengths to determine parameters. For example, more than two wavelengths and/or more than two structures may be analyzed sequentially or together.

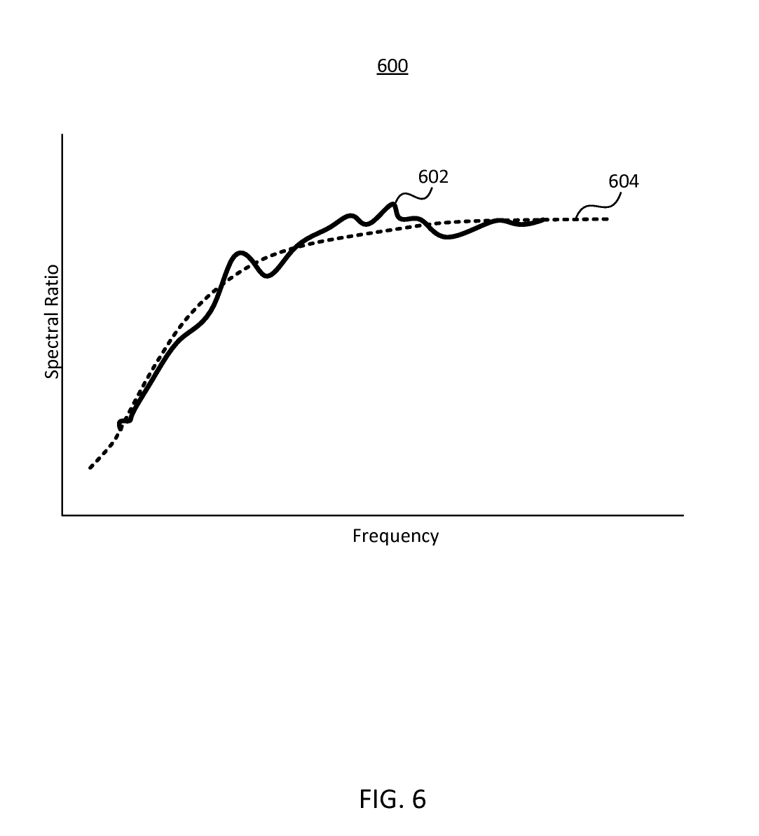

FIG. 6 is an illustrative plot 600 of the ratio of two frequency-domain photoacoustic signals in accordance with some embodiments of the present disclosure. The abscissa of plot 600 is presented in units of frequency, for example, in megahertz. The units of the ordinate of plot 600 correspond to spectral ratio of two detected photoacoustic signals.

Curve 602 of plot 600 may include points calculated from the ratio of two photoacoustic signals. In some embodiments, curve 602 may be the normalized spectral ratio of two photoacoustic signals. A normalized spectral ratio may include a simple ratio, a normalized ratio, dividing a signal by frequency, any other suitable calculations, or any combination thereof. For example, curve 602 may be determined by comparing curves 502 and 506 of FIG. 5. Curve 604 of plot 600 may be a calculated curve related to curve 602. In some embodiments, curve 604 may be calculated using Eq. 18, describing the ratio of two frequency-domain photoacoustic signals in terms of parameters including the ratio of their respective fluence and the absolute absorption coefficients at the signals' respective wavelengths. In some embodiments, the parameters used to calculate curve 604 may be adjusted to make curve 604 similar to curve 602. In some embodiments, determining curve 604 may include smoothing or averaging. For example, the data of curve 602 may be smoothed or averaged before parameters of an equation are adjusted to improve fitting. In some embodiments, parameters for Eq. 18 may be fit to smoothed or unsmoothed data related to the data of curve 602. The parameters may be adjusted using a multiple parameter fitting routine, a Monte Carlo analysis, any other suitable fitting method, or any combination thereof. The difference between the curves may be calculated and minimized using, for example, a least-squares calculation. In some embodiments, the system may extract a ratio of the fluences φ(λ₁)/φ(λ₂), and absorption coefficients μₐ_{₁}, μₐ_{₂} by fitting curve 604 to the data represented by curve 602. In some embodiments, curve fitting may include fitting the entire curve, portions of the curve, point-by-point analysis of the curve, one or more frequency elements contained within the curve, any other portion of the data, or any combination thereof. In some embodiments, the photoacoustic signals received from using a single wavelength used to measure a physiological change over time in a structure (e.g., a change in hemoglobin concentration) may have the same fluence, and thus the ratio of the fluences φ(λ₁)/φ(λ₂) may be 1. In some embodiments, the knowledge of the fluence ratio and the light wavelengths may be used in part to extract two absorption coefficients from the spectral ratio.

In some embodiments, light sources with different wavelengths may be used. Using different wavelengths, may result in a change in the ratio of the fluences φ(λ₁)/φ(λ₂). As described above, the ratio may be determined by fitting Eq. 18 or any other suitable equation to one or more curves. For example, the photoacoustic signals received from two wavelengths of light used to measure the same structure may result in two fluence values (and thus one fluence ratio) and two absorption coefficients. Other parameters may result in a non-unitary fluence ratio, for example, a change in position or configuration of light sources. In some embodiments, multiple frequency components in the frequency-domain photoacoustic spectra may result in multiple independent equations (e.g., Eq. 18) with multiple unknowns, which may be solved using any suitable computation technique. In some embodiments, signals may be compared using a point-by-point technique. In some embodiments, signals may be compared using a calculated curve function related to the signal data points.

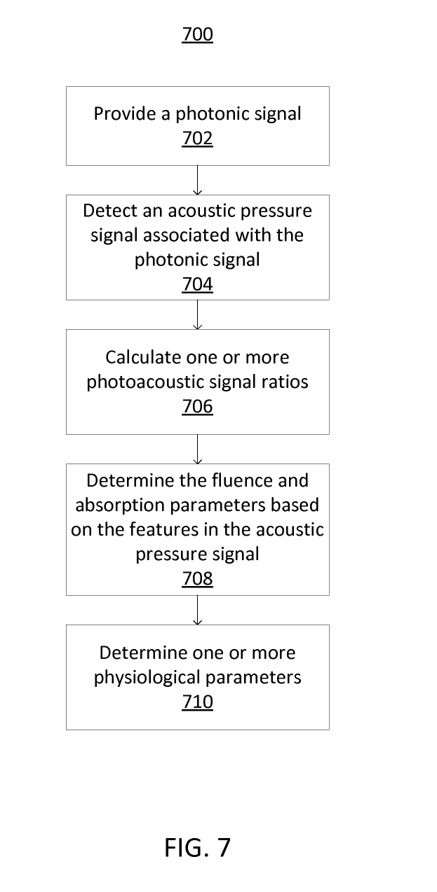

FIG. 7 is flow diagram 700 showing illustrative steps for determining a physiological parameter in accordance with some embodiments of the present disclosure.

In step 702, the system may provide one or more photonic signals from a light source. The light source may be, for example, light source 16 of FIG. 1, light source 502 of FIG. 5, any other suitable light source, or any combination thereof. In some embodiments, the light source may generate one or more particular wavelengths of light. In some embodiments, the light source may be one or more continuous wave laser diodes and the laser diodes may be modulated to generate a modulated photonic signal (e.g., a linearly-chirped frequency modulated signal). In some embodiments, the light source may produce a time-domain photoacoustic signal, a frequency-domain photoacoustic signal, any other suitable signal, or any combination thereof.

In step 704, the system may detect an acoustic pressure signal generated in the tissue of a subject in response to the one or more photonic signals from the light source. The acoustic pressure signal may be detected using an acoustic detector. The acoustic detector may, for example, be detector 18 of FIG. 1. The acoustic pressure signal may include a pressure signal generated as a result of the photoacoustic effect, as described above. The acoustic detector may include an ultrasonic detector or microphone capable of detecting the acoustic pressure signal. In some embodiments, the bandwidth of the signal may be determined in part by the bandwidth of the acoustic detector. The corresponding photoacoustic signal may include one or more components corresponding to the one or more photonic signals. It will be understood that components may also be referred to herein as separate signals. In some embodiments, the photoacoustic signal may be a processed version of the acoustic detector signal. For example, the photoacoustic signal may be derived based on an envelope detection performed on the acoustic detector signal.

In step 706, the system may calculate one or more photoacoustic signal ratios. In some embodiments, the system may perform processing steps on the acoustic signal before, after, or both before and after calculating one or more photoacoustic signal ratios. Processing steps may include domain transforms, normalization, baseline corrections, any other suitable processing steps, or any combination thereof. Domain transforms may include Fourier transforms. For example, a time-domain signal may be transformed to a frequency domain signal. In some embodiments, the system may perform certain processing steps in a first domain and other processing steps in a second domain. For example, the system may isolate a photoacoustic signal related to a particular structural element of a subject in a time domain, transform that signal to a frequency domain, and then calculate a spectral ratio in the frequency domain. Calculating the ratio may, for example, include dividing one signal by the other in the frequency domain. Calculating the ratio may include determining a spectral ratio. A spectral ratio may include a simple ratio, a normalized ratio, dividing a signal by frequency, any other suitable calculations, or any combination thereof. Normalization may include adjusting the amplitudes of a signal. Normalization may include rescaling steps, correction steps, any other amplitude adjustments, or any combination thereof. In some embodiments, normalization may include time or frequency corrections, for example, correcting for non-linearity of light sources and/or detectors. In some embodiments, an additional reference signal may be used for normalization. The reference may be generated using a calibration tool such as calibration device 80 of FIG. 1. In some embodiments, the system may use a reference sample with known optical properties for calibration. In some embodiments, the system may normalize a sample using peaks in the generated spectrum.

In step 708, the system may determine fluence and absorption parameters of the subject tissue based on the one or more photoacoustic signal ratios, for example the normalized spectral ratio, calculated in step 706. In some embodiments, a fluence ratio and absolute absorption coefficients may be determined using the methods described above in connection with Eqs. 15-18. For example, a normalized spectral ratio may be determined for pairs of photoacoustic signals generated by different wavelengths of light. The system may fit a curve to the normalized spectral ratio. The system may fit an equation or system of equations to the curves. The system may determine optical and physiological properties from fitting the curves. For example, the system may determine a fluence ratio of two wavelengths of light and an absorption coefficient at each wavelength for a given subject or element of a subject. It will be understood that these methods are merely exemplary and that the system may use any suitable methods for determining fluence and absorption parameters based on one or more photoacoustic signal ratios.

In some embodiments, the system may use the fluence ratio to calculate tissue properties, such as the blood oxygen saturation of tissue, for example, tissue peripheral to a target blood vessel in a subject. In some embodiments, the system may not use the fluence ratio to directly determine a physiological parameter, but it may be used in calculations for determining absorption coefficients and/or fitting curves. In some embodiments, the fluence ratio may include tissue attenuation coefficients, which may be related to the blood oxygen saturation of the tissue. In some embodiments, for example where the blood oxygen saturation of arterial blood within a relatively large blood vessel is the desired physiological parameter, the fluence ratio itself may be calculated for fitting but not be used in determining the blood oxygen saturation.

In some embodiments, the fluence and absorption parameters may be determined by fitting an equation to a photoacoustic signal ratio as illustrated in plot 600 of FIG. 6. In some embodiments, multiple photoacoustic signal ratios may be calculated and analyzed to determine the fluence and absorption parameters. For example, three or more acoustic signals associated with photonic signals of three or more wavelengths may be detected and multiple photoacoustic signal ratios may be calculated. The absorption coefficient for a particular wavelength may be determined based on an analysis of multiple photoacoustic signal ratios involving a photoacoustic signal associated with the particular wavelength. For example, an initial absorption coefficient for the particular wavelength may be determined for each ratio and then the initial absorption coefficients may be averaged together to determine a final absorption coefficient. As another example, the photoacoustic signal ratios may be analyzed and one or more ratios may be selected to be used to determine fluence and absorption parameters. The analysis may include determining a curve fit error, determining a signal quality of one or more photoacoustic signals, any other suitable metric calculation, and any suitable combination thereof.

In step 710, the system may determine one or more physiological parameters. The system may determine the one or more physiological parameters based on the one or more photonic signals emitted in step 702, the one or more acoustic pressure signals received in steps 704, one or more absorption coefficients determined in step 708, and one or more fluence ratios determined in step 708. The physiological parameters may be determined using the methods described above in connection with Eqs. 19-23. It will be understood that these methods are merely exemplary and that the system may use any suitable methods for determining physiological parameters. In some embodiments, the system may use information from other sources to determine physiological parameters including lookup tables, user inputs, information from other sensors, any other suitable information, or any combination thereof.

In some embodiments, in step 710 the system may determine a temperature. For example, the system may determine the internal temperature of a subject using a photoacoustic signal. The system may use the absorption coefficients determined in step 708 and the received photoacoustic signal or signals to determine a Grüneisen parameter Γ using, for example, Eq. 1. A temperature may be determined from the Grüneisen parameter Γ using, for example, a lookup table or conversion equation.

In some embodiments, in step 710 the system determine physiological parameters such as hemoglobin concentration (e.g., oxygenated, deoxygenated, total, or a combination thereof), blood oxygen saturation (e.g., arterial, venous, or both), temperature, cardiac output (CO), intrathoracic blood volume (ITBV), intrathoracic circulatory volume (ITCV), global end-diastolic volume (GEDV), pulmonary circulatory volume (PCV), extravascular lung water (EVLW), any other suitable hemodynamic parameters, any other suitable physiological parameters, any physiological modulations thereof, or any combination thereof.

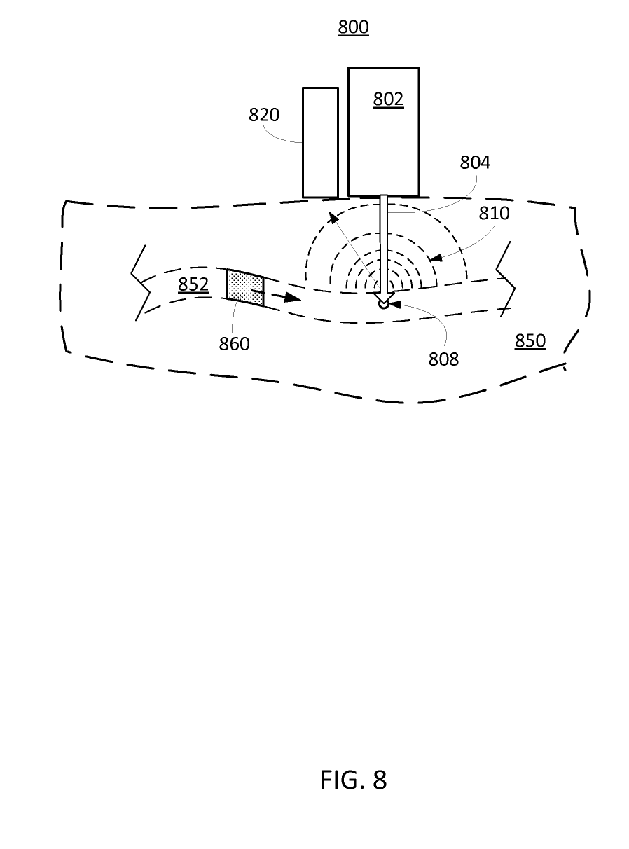

FIG. 8 shows an illustrative indicator dilution photoacoustic arrangement 800, in accordance with some embodiments of the present disclosure. Indicator dilution photoacoustic arrangement 800 may be used to determine, for example, cardiac output (CO), intrathoracic blood volume (ITBV), intrathoracic circulatory volume (ITCV), global end-diastolic volume (GEDV), pulmonary circulatory volume (PCV), extravascular lung water (EVLW), and/or other hemodynamic parameters as described further below. In some embodiments, these parameters may be determined based on temperature measurements determined using the techniques described above.

Light source 802 may provide photonic signal 804 to subject tissue 850 including blood vessel 852. Photonic signal 804 may be attenuated along its path length by subject tissue 850 prior to reaching target area 808 for blood vessel 852. It will be understood that photonic signal 804 may scatter in subject 850 and need not travel in a well-formed beam as illustrated. Also, photonic signal 804 may generally travel through and beyond blood vessel 852. A constituent of the blood in blood vessel 852 such as, for example, hemoglobin, may absorb at least some of photonic signal 804. Accordingly, the blood may exhibit an acoustic pressure response resulting in acoustic pressure signal 810 via the photoacoustic effect, which may act on the surrounding tissues of blood vessels 852. Acoustic pressure signal 810 may be detected by acoustic detector 820. In some embodiments, changes in some properties of the blood in blood vessel 852 at site 808 may be detected by acoustic detector 820 as a change in photoacoustic signal 810. For example, a reduced hemoglobin concentration or reduced temperature at the monitoring site may cause a reduced acoustic pressure signal to be detected by acoustic detector 820. In some embodiments, bolus dose 860, which may include a suitable indicator, may be introduced to the blood of patient 850 at a suitable blood vessel site (not shown in FIG. 8). Acoustic detector 820 may detect the transient changes in the hemoglobin concentration (“hemo-dilution”) and/or temperature (“thermo-dilution”) at site 808 due to passage of bolus dose 860 through site 808. In some embodiments, multiple monitoring sites (not shown) may be used to detect changes in hemoglobin concentration, temperature, or both. As bolus dose 860 travels through the circulatory system of subject 850, diffusion, mixing (e.g., within a heart chamber), or both may spread the hemoglobin concentration and temperature profiles axially (i.e., in the direction of flow) and radially (i.e., normal to the direction of flow). It will be understood that hemo-dilution refers to the dilution of blood constituents caused by the bolus dose, and thereto-dilution refers to the combined effects of blood constituent dilution and temperature change, both caused by the bolus dose. In some embodiments, using a thereto-dilution indicator, a temperature change may be enhanced by hemo-dilution (e.g., when the temperature change and the dilution change both cause the photoacoustic signal to either increase or decrease), and accordingly may be detected by a system having relatively less temperature sensitivity.

It will be understood that light source 802 may correspond to light source 402 of FIG. 4. For example, light source 802 may include a pulsed laser diode which may provide a high signal to noise ratio photoacoustic signal. As a further example, light source 802 may include a continuous wave laser diode or any other suitable light source. It will also be understood that arrangement 800 may be included as part of system 10 of FIG. 1, sensor unit 12 of FIG. 2 or system 300 of FIG. 3.

A bolus dose of an indicator may cause the properties at a photoacoustic monitoring site to change in time as the bolus dose passes the site. Introduction of the indicator may alter one or more properties of the blood that interacts with the indicator (e.g., blood near the bolus dose). An indicator introduced as a bolus dose may be selected to have one or more properties that allow the bolus dose to be distinguished from a subject's un-dosed blood. For example, an indicator may be selected which has particular absorption properties at one or more particular wavelengths (e.g., a dye indicator such as indocyanine green dye), and the photoacoustic monitoring system may monitor the presence of the indicator by providing a photonic signal at one or more particular wavelengths and detecting an acoustic pressure signal having a dye indicator dilution response. In a further example, an indicator may be selected to dilute blood of a subject but not substantially absorb the photonic signal. The photoacoustic monitoring system may then accordingly monitor the blood (e.g., hemoglobin) rather than the indicator, to detect dilution. In a further example, an indicator having a temperature different from the temperature of the subject's un-dosed blood may be introduced into a subject's bloodstream (e.g., a “hot” or “cold” indicator, relative to the blood temperature). The photoacoustic monitoring system may then accordingly monitor the bloodstream temperature at the monitoring site, or the combined effects of hemo-dilution and thermo-dilution achieved by the bolus dose. In some embodiments, an indicator may have more than one property that may be distinguished from a subject's blood. For example, a cold dye indicator may be introduced to the subject's bloodstream, which may allow hemo-dilution and thereto-dilution effects to be detected. In some embodiments, more than one indicator may be introduced to the subject's bloodstream, each indicator having particular properties that may be unique relative to the other indicators. For example, an isotonic indicator and a hypertonic indicator may be introduced into a subject's bloodstream. In a further example, a cold isotonic indicator and a dye indicator may be introduced into a subject's bloodstream. An indicator may include saline (e.g., isotonic, hypertonic, hypotonic), dye (e.g., indocyanine), lithium, any other suitable chemical or mixture, or any combination thereof.

In some embodiments, a relatively small amount of indicator may be introduced to a subject's bloodstream. For example, a bolus dose on the order of 10 mL may be injected to act as an indicator. Accordingly, the detected response may be relatively small. For example, the temperature change caused by a thereto-dilution indicator may be less than 1 degree Celsius, depending on the amount of indicator used and the monitoring arrangement used.

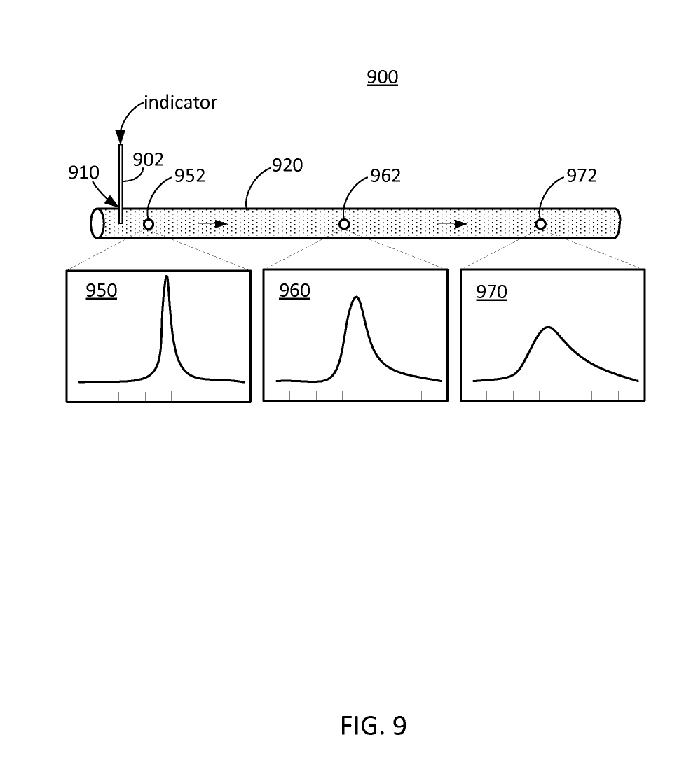

FIG. 9 shows an illustrative indicator arrangement 900, in accordance with some embodiments of the present disclosure. In some embodiments, an indicator may be provided to the circulatory system of a subject to aid in determining one or more physiological parameters. For example, a saline solution may be injected into a subject's circulatory system at blood vessel site 910 using needle 902. Blood vessel site 910 may be located at any suitable portion of a subject's circulatory system such as a vein, an artery, a capillary, or other suitable location. For example, blood vessel site 910 may be a central vein of the subject. Portion 920 of the subject's circulatory system shown illustratively in FIG. 9 may include heart chambers, arteries, veins, capillaries, any other suitable parts of the circulatory system, or any combination thereof. As the indicator travels along portion 920, in the direction of the motion arrows away from the introduction site, the concentration and/or temperature profile of may change. For example, panel 950 shows an illustrative dilution curve time series as detected at site 952, relatively near site 910. Panels 960 and 970 each show illustrative time series of dilution curves at sites 962 and 972, respectively, both downstream from site 952. The dilution curve shown in panel 960 is relatively flattened in time compared to the dilution curve shown in panel 950. The dilution curve shown in panel 970 is relatively flattened in time compared to the dilution curve shown in panel 960. The flattening may be due to diffusion and mixing of the indicator with the subject's blood. The area under the time series of panels 950, 960, and 970 may be, but need not be, the same and may depend on the indicator type, travel time, site location, and other suitable variables. The phrase “dilution curve” as used herein shall refer to a time series, continuous or discrete, indicative of dilutive effects of an indicator on the concentration of blood constituents and/or blood temperature. For example, a dilution curve may include a time series of concentration or changes thereof of a blood constituent, an indicator, or both. In a further example, a dilution curve may include a time series of temperature, or change in temperature, of blood of the subject at a monitoring site. As the indicator is transported through the subject's vasculature, a portion of the indicator may travel through each blood vessel, proportional to the flow rate of blood in that vessel. Accordingly, the original bolus dose of indicator may “mix out” after some time, and a steady-state, or near steady-state condition may be achieved (e.g., similar to a steady-state or near steady-state condition before the bolus dose was introduced).

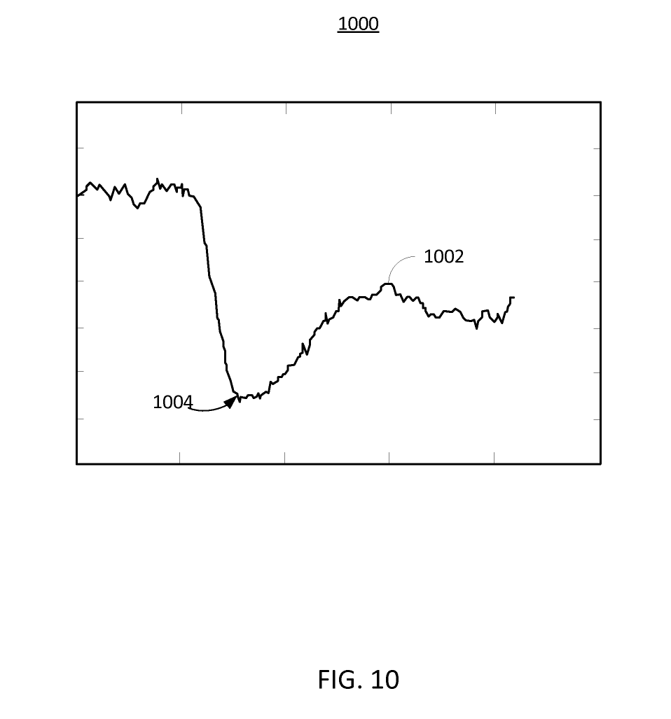

FIG. 10 shows a plot 1000 of an illustrative photoacoustic signal 1002, including a response corresponding to an isotonic indicator (e.g., 0.9% w/v saline), in accordance with some embodiments of the present disclosure. A light source is used to provide a photonic signal to a first site of a circulatory system, causing a photoacoustic response of constituents in the circulating blood at that site. An acoustic detector is used to detect acoustic pressure signals caused by the photonic signal at the first site, and along with processing equipment, output a corresponding photoacoustic signal. An isotonic indicator is injected as a bolus dose into the circulating blood at a second site. As the bolus dose travels past the first site, the hemoglobin concentration at the first site temporarily decreases. Trough 1004 indicates the dilatory effects of the bolus dose of isotonic indicator. The processing equipment outputs a reduced photoacoustic signal caused by the reduced hemoglobin concentration. The effect of the indicator may be detected as a trough in the photoacoustic signal corresponding to the passing of the bolus dose through the first site. Note that photoacoustic signal 1002 may be indicative of tHb, exhibiting a substantially steady-state baseline and trough 1004 indicative of the presence of the indicator (e.g., a reduction of tHb via displacement by the indicator). Note that a plot of indicator concentration as a function of time may exhibit a peak, corresponding to a steady-state tHb value minus an instantaneous tHb value. A dilution curve may include either a peak or a trough depending upon the species monitored and the units used in calculation.

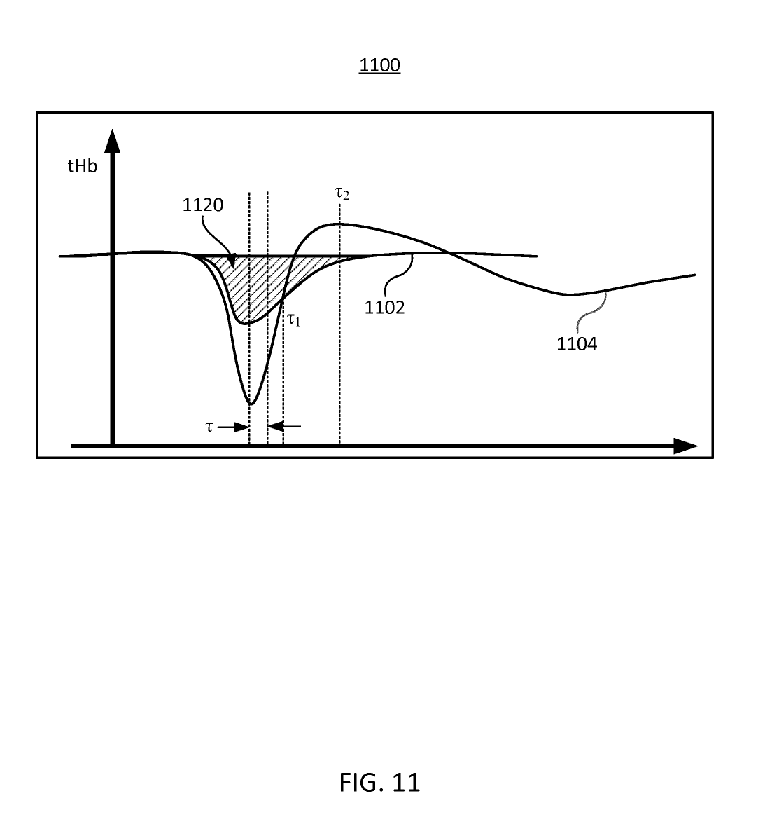

FIG. 11 shows an illustrative plot 1100 of two total hemoglobin concentration time series as respective isotonic and hypertonic indicators pass a photoacoustic detection site, in accordance with some embodiments of the present disclosure. The abscissa of plot 1100 is shown in units of time, and the ordinate of plot 1100 is shown in units of total hemoglobin concentration, although any suitable units may be used in accordance with the present disclosure. Time series 1102 and 1104 may be pre-processed and/or processed signals derived from the output of an acoustic detector. For example, time series 1102 and 1104 may each include sample points corresponding to a maximum acoustic response for each light pulse (e.g., an acoustic pressure peak value), at a particular time lag corresponding to a particular spatial location (e.g., a particular blood vessel). Time series 1102 is total hemoglobin concentration as the isotonic indicator travels through the photoacoustic detection site. Alternatively, if the indicator concentration were shown rather than tHb, it may exhibit a peak rather than a trough, corresponding to the shaded area 1120. Time series 1104 is total hemoglobin concentration as the hypertonic indicator travels through the photoacoustic detection site. The variable t as shown in FIG. 11 represents time relative to each response, and not an absolute time scale. For example, the time origin for both responses may be zero, and they may be plotted on the same axis even though the indicators were introduced at different times. The variable x as shown in FIG. 11 represents the mean transit time difference between the two responses.

In some embodiments, one or more characteristics may be derived from one or both responses. For example, the flow rate of a particular indicator may be formulated as shown by:

\dot{V}Cᵢ=\dot{N} (24)

where \dot{V} is the volumetric flow rate of blood (e.g., volume/time, assumed here to be constant in time), Cᵢ is the concentration of indicator i (e.g., mole/volume), and \dot{N} is the molar flow rate of molecule i (e.g., mole/time). Defining the cardiac output CO to be equal to volumetric flow rate \dot{V}, and referencing time series 1102, the following Eq. 25 may be derived by integrating both sides of Eq. 24 in time:

$$CO = \frac{N}{A} \qquad ( 25 )$$

where cardiac output CO is proportional to the total isotonic indicator amount introduced N (e.g., moles), and A is given by:

A=∫Cᵢdt (26)

where A may be equivalent to the area 1120 bounded by time series 1102 and the steady tHb value. Under some circumstances, cardiac output may be equal to the ratio of isotonic indicator amount introduced and the area bounded by the time series and the steady tHb value, while in other circumstances the equality of Eqs. 15-16 may be replaced by the proportionality symbol ∝ (e.g., to account for density differences). Area A is an illustrative example of a characteristic derived from a response to an indicator.

In a further example, EVLW may be determined based on isotonic and hypertonic indicators, as shown by Eq. 27:

EVLW=CO*ΔτC_{MT} (27)

where CO is the cardiac output, and Δτ_{MT} is the mean transit time difference between the isotonic and hypotonic indicator dilution curves. The mean transit time of an indicator dilution curve may be based on any suitable reference point of the curve. The mean transit time for a dilution curve may be calculated using Eq. 28:

$${τ}_{MT} = {τ}_{0} + \frac{\int {C}_{i} * ( t - {τ}_{0} ) d t}{\int {C}_{i} d t} \qquad ( 28 )$$

where τ₀ is the time after introduction of the indicator when the indicator is detected at the PA monitoring site, and Cᵢ is the indicator concentration.

In a further example, a vascular permeability metric vp may be defined as:

vp=τ₂−τ₁ (29)

where τ₂ is the time corresponding to a trough (i.e., minimum, occurring after a peak) of the response to the hypertonic indicator, and τ₁ is the time where the responses to the isotonic and hypertonic indicators cross. In some circumstances, vascular permeability may provide an indication and/or measure of the possibility of a capillary leak and the possibility of fluid accumulating outside of the blood vessels.

In a further example, EVLW may be determined based on an osmotic response (e.g., the transfer of water and salt between the blood and lungs due to a chemical potential difference) of the subject using an isotonic and hypertonic indicator. EVLW may be determined using the following Eq. 30, for the hypertonic indicator:

$$EVLW = \frac{{Π}_{b} ( \frac{Δ {n}_{3}}{c} - Δ {EVLW}_{3} )}{Δ {Π}_{b , 3}} \qquad ( 30 )$$

where Π₄, is the steady state osmolarity of the subject's blood (e.g., before introduction of the hypertonic indicator), ΔΠ_{b,3} is the change in the osmolarity of subject's blood at time Σ₃, Δn₃ is the total amount of salt transferred from the subject's blood to the subject's lungs at time τ₃, c is the concentration of solutes in the EVLW, and ΔEVLW₃ is the total change in extravascular lung water at time τ₃. The time τ₃ is the time, referenced to zero at the beginning of the response, when the EVLW and blood have the same osmotic pressure for the hypertonic indicator.

In some embodiments, a thermo-dilution indicator may be introduced to the subject's circulatory system at a suitable location. For example, in some embodiments, a saline solution having a temperature less than that of a subject's blood may be introduced, and one or more dilution curves may be measured at one or more respective locations in the subject's vasculature. The Grüneisen parameter of the subject's blood may depend on temperature linearly according to:

Γ=mT+b (31)

where m is a slope and b is an intercept. Accordingly, Eq. 1 may be rewritten as follows:

p(z,T)=Γ(T)μₐφ(z) (32)

Showing that as the temperature at the photoacoustic monitoring site changes, the acoustic pressure signal and a photoacoustic signal derived thereof may change accordingly. Introduction of thereto-dilution indicator may be used to determine cardiac output, ITCV, PCV, and/or GEDV, for example.

In some embodiments, cardiac output CO may be calculated using:

$$CO = K \frac{( {T}_{b , 0} - {T}_{i , 0} ) {V}_{i}}{\int ( {T}_{b , 0} - {T}_{b} ( t ) ) d t} \qquad ( 33 )$$

where K is a proportionality constant (e.g., including the effects of specific gravity and heat capacity of blood and/or the indicator), T_{b,0} is the initial blood temperature at the time and site of injection, T_{i,0} is the initial indicator temperature, Vᵢ is the volume of injected indicator, and T_{b}(t) is the blood temperature at time t, as measured using the photoacoustic technique. Note that the moles of injected indicator may be used rather than Vᵢ in some cases, with a suitable adjustment of the proportionality constant K to include the indicator concentration (e.g., mole/volume).

In some embodiments, ITCV may be calculated using:

ITCV=CO*τ_{MT} (34)

where CO is the cardiac output, and T_{MT} is the mean transit time of the thermo-dilution curve. The mean transit time for a thermo-dilution indicator may be calculated using:

$${τ}_{MT} = {τ}_{0} + \frac{\int ( {T}_{b , 0} - {T}_{b} ( t ) ) * ( t - {τ}_{0} ) d t}{\int ( {T}_{b , 0} - {T}_{b} ( t ) ) d t} \qquad ( 35 )$$

where τ₀ is the time after introduction of the indicator when the indicator is detected at the PA monitoring site, and (T_{b,0}−T_{b}(t)) is the difference in initial and instantaneous blood temperature of the thermo-dilution curve. In some embodiments, in which a thermo-dilution indicator may be used, a circulatory volume may be equivalent to a thermal volume.

In some embodiments, PCV may be calculated using:

PCV=CO*τ_{DS} (36)

where CO is the cardiac output, and τ_{DS} is the downslope time of the thermo-dilution curve. In some embodiments, the downslope time may be determined as the time interval of the linear decay of the indicator response (e.g., downslope of a peak), from about 80% of the peak value to about 20% of the peak value. In some circumstances, downslope time may provide an indication and/or measure of the washout of the indicator, which may depend on the volume which the indictor dilutes.

In some embodiments, GEDV may be calculated using:

GEDV=ITCV−PCV (37)

which may be indicative of the blood volume included in the ITCV.

In some embodiments, EVLW may be calculated using:

EVLW=ITCV−ITBV (38)

where ITBV may be calculated from GEDV, which may be calculated using Eq. 37. For example, ITBV may be directly proportional to GEDV, with a proportionality constant of order one (e.g., a constant of 1.25).

In some embodiments, more than one thermo-dilution indicator may be introduced to a subject. For example, two thermo-dilution indicators, at two different temperatures, may be introduced to the subject. Differences in the resulting dilution curves may provide information regarding hemo-dilution, thermo-dilution, or differences thereof.

In some embodiments, both a thermo-dilution indicator and a hemo-dilution indicator may be introduced to the subject's circulatory system at suitable locations and times. For example, in some embodiments, a saline solution having a temperature less than that of a subject's blood may be introduced, and a dye indicator such as indocyanine green dye may be introduced. Accordingly, two or more dilution curves may be measured at one or more locations in the subject's vasculature, indicative of the hemo-dilution and thereto-dilution indicators. Any of the properties that may be calculated using Eqs. 31-37 may be calculated using the thermo-dilution indicator. In some embodiments, ITBV may be calculated using the hemo-dilution curve, as shown by:

ITBV=CO*τ_{MT} (39)

where CO is the cardiac output (e.g., calculated using Eq. 25 or 33), and τ_{MT} is the mean transit time of the hemo-dilution curve (e.g., calculated using Eq. 28).

In some embodiments, EVLW may be calculated from the thermo-dilution curve and hemo-dilution curve using:

EVLW=ITCV−ITBV (40)

wherein ITCV may be calculated from the thermo-dilution curve (e.g., using Eq. 34), and ITBV may be calculated from the hemo-dilution curve (e.g., using Eq. 39).

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed is:

1. A photoacoustic system for determining a physiological parameter of a subject, the system comprising:
at least one frequency modulated light source configured to provide a photonic signal to the subject;
at least one acoustic detector configured to detect an acoustic pressure signal from the subject, wherein the acoustic pressure signal is caused by absorption of at least some of the photonic signal by the subject; and
processing equipment configured to:
calculate a plurality of spectral ratios based on the acoustic pressure signal,
determine at least one optical absorption coefficient based on the plurality of spectral ratios, and
determine the physiological parameter based on the at least one optical absorption coefficient.

2. The system of claim 1, wherein the processing equipment is further configured to:
fit the plurality of spectral ratios to a function; and
determine the at least one optical absorption coefficient based on the fitted function.

3. The system of claim 1, wherein the processing equipment is further configured to:
process the acoustic pressure signal to generate a time domain signal;
identify elements in the time domain signal;
extract the identified time domain elements from the time domain signal; and
calculate a frequency domain signal based on the isolated time domain elements.

4. The system of claim 1, wherein the at least one frequency modulated light source comprises at least one laser diode.

5. The system of claim 1, wherein the at least one frequency modulated light source is modulated using a linear chirp frequency modulation.

6. The system of claim 1, wherein the at least one acoustic detector comprises a piezoelectric ultrasound detector.

7. The system of claim 1, wherein the plurality of spectral ratios comprise ratios of frequency components greater than 1 megahertz.

8. The system of claim 1, wherein the processing equipment is further configured to determine a fluence ratio.

9. The system of claim 1, wherein the plurality of spectral ratios are a plurality of normalized spectral ratios.

10. The system of claim 1, wherein the physiological parameter is selected from the group comprising hemoglobin concentration, blood oxygen saturation, temperature, cardiac output (CO), intrathoracic blood volume (ITBV), intrathoracic circulatory volume (ITCV), global end-diastolic volume (GEDV), pulmonary circulatory volume (PCV), extravascular lung water (EVLW), and any suitable combination thereof.

11. A photoacoustic method for determining a physiological parameter of a subject, the method comprising:
providing a photonic signal to the subject from at least one frequency modulated light source;
detecting an acoustic pressure signal from the subject using at least one acoustic detector, wherein the acoustic pressure signal is caused by absorption of at least some of the photonic signal by the subject;
calculating, using processing equipment, a plurality of spectral ratios based on the acoustic pressure signal;
determining, using the processing equipment, at least one optical absorption coefficient based on the plurality of spectral ratios; and
determining, using the processing equipment, the physiological parameter based on the at least one optical absorption coefficient.

12. The method of claim 11, the method further comprising:
fitting, using the processing equipment, the plurality of spectral ratios to a function; and
determining, using the processing equipment, the at least one optical absorption coefficient based on the fitted function.

13. The method of claim 11, the method further comprising:
processing, using the processing equipment, the acoustic pressure signal to generate a time domain signal;
identifying, using the processing equipment, elements in the time domain signal;
extracting, using the processing equipment, the identified time domain elements from the time domain signal; and
calculating, using the processing equipment, a frequency domain signal based on the isolated time domain elements.

14. The method of claim 11, wherein providing a photonic signal to the subject from at least one frequency modulated light source further comprises providing light from at least one laser diode.

15. The method of claim 11, wherein providing a photonic signal to the subject from at least one frequency modulated light source further comprises providing a linear chirp frequency modulation.

16. The method of claim 11, wherein detecting an acoustic pressure signal from the subject using at least one acoustic detector further comprises detecting using a piezoelectric ultrasound detector.

17. The method of claim 11, wherein calculating, using the processing equipment, the plurality of spectral ratios based on the acoustic pressure signal further comprises calculating ratios of frequency components greater than 1 megahertz.

18. The method of claim 11, the method further comprising determining, using the processing equipment, a fluence ratio.

19. The method of claim 11, wherein calculating, using the processing equipment, the plurality of spectral ratios based on the acoustic pressure signal comprises calculating a plurality of normalized spectral ratios.

20. The method of claim 11, wherein determining, using the processing equipment, the physiological parameter based on the at least one optical absorption coefficient comprises determining a physiological parameter selected from the group comprising hemoglobin concentration, blood oxygen saturation, temperature, cardiac output (CO), intrathoracic blood volume (ITBV), intrathoracic circulatory volume (ITCV), global end-diastolic volume (GEDV), pulmonary circulatory volume (PCV), extravascular lung water (EVLW), and any suitable combination thereof.
